(19) United States
(12) Patent Application Publication
(10) Pub. No.: US 2010/0291177 A1
(43) Pub. Date: Nov. 18, 2010

(54) PREVENTION OF STAPHYLOCOCCUS BIOFILM FORMATION

(76) Inventors: Peter Wilhelmus Maria Hermans, Huissen (NL); Johan Van Eldere, Leuven (BE)

Correspondence Address: JACKSON WALKER LLP, 901 MAIN STREET, SUITE 6000, DALLAS, TX, 75202-3797, US

(21) Appl. No.: 12/595,657

(22) PCT Filed: Apr. 11, 2008
(86) PCT No.: PCT/NL08/50206

§ 371 Date: Apr. 23, 2010

(30) Foreign Application Priority Data
Apr. 13, 2007 (EP) 07106164.2

(51) Int. Cl. A61K 9/00; A61K 39/40; A61K 39/395; A61K 39/02; A61P 31/04; A61P 37/04

(52) U.S. Cl. 424/423; 424/165.1; 424/422; 424/135.1; 424/133.1; 424/190.1

(57) ABSTRACT

The present application discloses antibodies directed to protein SE2232 (SesC) of Staphylococcus epidermis, and homologus proteins which are useful in preventing biofilm formation by said micro-organism. The antibodies can be used to coat medical devices which are inserted or implanted into the mammalian, preferably human body. Further these antibodies or vaccines comprising said proteins can be used to vaccinate subjects to prevent or treat natural occurring biofilm formation during infection. Also covered are medical devices coated with the antibody.

The invention relates to the field of medical microbiology, more specifically the prevention of biofilm formation by bacteria, more specifically wherein said bacteria are Staphylococcus genus bacteria.

### BACKGROUND OF THE INVENTION

Staphylococcus epidermis and other coagulase-negative staphylococci (CONS) are the leading cause of foreign-body infections (FBI), i.e. infections which occur on devices which have been inserted or implanted in the body, such as (but not limited to) intravascular devices (stents), prosthetic valves, endovascular grafts, orthopaedic endoprotheses and cerebrovascular shunts. For prosthetic valve endocarditis, 40-50% of the infections are due to S. epidermis, while this bacterial species is responsible for 50-70% of catheter-related infections (van Eiff, C. et al., 2001, Postgrad. Med. 110(4):63-76). In the United States alone, it is estimated that infections caused by S. epidermidis cost the public health-system at least $1 billion/year (Yao, Y. et al., 2005, J. Infect. Dis. 191:289-298).

The presence of foreign bodies facilitates the transition of S. epidermidis from a commensal microorganism to an opportunistic pathogen (Gotz, F. and Peters, G., 2000, In: Waldvogel, F. A. and Bisno, A. L. (ed.) Infections associated with indwelling medical devices. ASM Press, Washington D.C.). Biofilm formation on the surface of foreign bodies is a key factor in this process (Christensen, G. D. et al., 1982, Infect. immune. 37:318-326).

The development of a biofilm occurs in several steps. Initial attachment can be based on direct binding to the abiotic surface of an implant. This process is mediated by several proteins and carbohydrate factors (e.g. staphylococcal surface proteins SSP-1, SSP-2 (Veenstra, G. J. et al., 1996, J. Bacteriol. 178:537-541), capsular polysaccharide adhesin (PS/A) (Mack, D., 1999, J. Hosp. infect. 43(Suppl.):S113-S125; Shiro, H. et al., J. Infect. Dis. 169:1042-1049)). Alternatively, indirect binding to the surface of implanted medical devices coated with an absorbed layer of blood plasma proteins such as fibrinogen, fibronectin, collagen, vitronectin and elastin can be mediated via cell-wall-associated (CWA) proteins SdrG (The) (Davis, S. L. et al., 2001, J. Biol. Chem. 276:27799-27805; Nilsson, M. et al., 1998, Infect. immune. 66:2666-2673), Embp (Williams, R. J. et al., 2002, Infect. immune. 70:6805-6810), GehD (Bowden, m. G. et al., 2002, J. Biol. Chem. 277:43017-43023), AtlE (Heilmann, C. et al., 1997, Mol. Microbiol. 24:1013-1024) and Ebps (Park, P. W. et al., 1996, J. Biol. Chem. 271:15803-15809), respectively.

In a second phase, the bacteria proliferate and accumulate into multilayered cell clusters that are embedded in an extracellular material. Bacterial proliferation and production of extracellular matrix molecules lead to a more structured biofilm.

The icaADBC operon mediates the cell clustering and synthesis of the staphylococcal polysaccharide intercellular adhesin (PIA) (von Eiff, c. et al., 2002, Lancet Infect. Dis. 2:677). PIA facilitates intercellular adhesion and plays an essential role in cell accumulation. PIA has been identified as an important virulence factor of S. epidermidis (Rupp, M. E. et al., 1999, Infect. Immun. 67:2627-2632; Rupp, M. E. et al., 1999, Infect. Immun. 67:2656-2659). Proteins also seem to be essential for accumulation. The S. aureus Bap homologue protein, (Bhp) has been proposed to promote primary attachment to abiotic surfaces, as well as intercellular adhesion during biofilm formation (Cucarella, C. et al., 2001, J. Bacterial. 183:2888-2896). The accumulation-associated protein (Aap) is thought to play an essential role in cell accumulation, independent of PIA (Hussain, M. et al., 1997, Infect. Immun. 65:519-524). It has been shown that polyclonal and monoclonal antibodies against Aap can significantly reduce S. epidermidis accumulation in vitro (Hussain, m et al., supra; Sun, D. et al., 2005, Clin. Diagn. Lab. Immunol. 12:93-100).

Detachment of cell clusters from an existing biofilm can occur, and this is attributed to the expression of a class of peptides known as phenol-soluble modulins (Yao, Y. et al., supra, Otto, M. et al., 2004, J. Infect. Dis. 190:748-755). However, our understanding of the mechanism of the detachment process is very limited.

Because most infections due to caogulase-negative staphylococci are nocosomial, it is not surprising that these infections have become increasingly resistant to multiple antibiotics: 80-90% of the isolated strains shows inducible beta-lactamase activity, 80% are methicillin-resistant (von Eiff, C. et al., 2001, Postgrad. Med. 110:63-76). Further, bacteria within the biofilm are tolerant to antibiotics and antimicrobial agents. It is estimated that sessile bacteria in biofilms are 1,000 to 1,500 times more resistant to antibiotics than their planktonic counterparts (Costerton, J., 1999, J. Antimicrob. Agents 11:217-221). This antibiotic resistance of biofilms often leads to the failure of conventional antibiotic therapy and necessitates removal and reinsertion of the infected device (Stewart, P. S. and Costerton, J. W., 2001 Lancet 358:135-138; Hoyle, B. D. and Costerton, J. W., 1991, Prog. Drug Res. 37:91-105, von Eiff, C. et al., Lancet Infect. Dis. 2:677-685 and Vuong, C. and Otto, M, 2002, Microbes Infect. 4:481-489).

The traditional approach to prevent biofilm formation in vivo is local or systemic administration of bactericidal agents (Danese, P. N., 2002, Chem. Biol. 9:873-880). Targeting specific staphylococcal biofilm-associated virulence factors, such as the formation of a slime capsule, is a novel approach for the treatment of staphylococcal infections (Vuong, C. and Otto, M, supra). Antibodies against extracellular macromolecules and surface binding proteins essential for cell-surface and cell-cell interaction adhesion, such as NA, teichoic acids, Fbe and Aap have been shown to prevent biofilm formation without killing the bacteria (e.g. Bowden, M. G. et al., 2005, Microbiology 151:1453-1464).

Yet, there still exists need for better or alternative therapy for preventing formation of biofilms.

### SUMMARY OF THE INVENTION

The invention now relates to an antibody against protein SE2232 of Staphylococcus epidermis and antibodies against proteins from Staphylococcus spp. which are homologous with said SE2232 protein. Said antibodies are preferably selected from the group consisting of polyclonal antibodies, monoclonal antibodies, single chain antibodies, humanised antibodies and SE2232 binding antibody fragments such as F(ab), F(ab)2 and scFV.

The invention further comprises a method for preventing biofilm formation by Staphylococcus on a medical device comprising coating said medical device with an antibody according to the invention. Said medical device is preferably an intravascular device such as a stent or a cannula, a prosthesis, such as an orthopaedic endoprothesis or a prosthetic valve, a pacemaker, a catheter, a drain, an endovascular graft or a cerebrovascular shunt. This medical device is to be implanted or inserted into a mammalian body, preferably a human body.

A further embodiment of the invention is the use of an antibody according of the invention for preventing biofilm formation, preferably wherein the antibody is coated onto a medical device. Also comprised within the invention is a method to prevent biofilm formation in a mammal in vivo and the use of the antibodies of the invention for such a method.

### DESCRIPTION OF THE FIGURES

FIG. 1. In vitro expression of genes SE1501 (1A) and SE2232 (1B) in sessile versus planktonic bacteria after inoculation in 0.9% NaCl. Bacteria in end-exponential growth phase were suspended in 0.9% NaCl with catheter fragments at time 0 min. Gene expression is quantified as the log₁₀ cDNA/gDNA ratio on the Y axis. The error bars indicate standard errors. The X axis indicates time (hours).

FIG. 2. Evolution of the in vivo expression of genes SE1501 and SE2232 during the first 24 hours after implantation (A) and between 24 hours and 2 weeks (B). The expression level at any time point is represented as the log 10 cDNA/gDNA ratio (Y axis) versus time (in minutes (A) and hours (B)) on the X axis. The error bars indicate standard errors.

FIG. 3. Images taken by fluorescence microscopy from planktonic (P) and sessile (S) bacteria at different time points for SE1501 (I) and SE2232 (II). Images A1 and A2 shows bacterial cells plus control rabbit antibody and FITC-labelled goat-anti-rabbit antibody visualized by fluorescence (A1) and yellow light (A2). IB1 and IB2 images were taken from control samples containing only bacterial cells plus RPAbSE1501 and IIB1 and IIB2 images of bacterial cells plus RPAbSE2232. Images of C1 and C2 were taken from control sample of cells plus only FITC-labelled goat-anti-rabbit antibody visualized by fluorescence and yellow light.

Cells from sessile samples are in a more condensed and aggregated condition. In the images of planktonic samples for SE1501 we can see that protein expression of SE1501 in the first 30 min of the experiment decreased and then increased and peaked at the end of experiment. In images taken from sessile samples it is clear that the expression of protein SE1501 gradually increased over time. In the images of planktonic samples for SE2232, it can be observed that the expression of SE2232 decreased at the beginning of the experiment and increased in samples taken at 90 and 120 min. In images taken from sessile samples we can see that expression of SE2232 after a decrease at 30 min increased again.

Expressions of both proteins (SE1501 and SE2232) in all sessile samples were higher than in their planktonic counterpart samples.

FIG. 4. S. epidermis 10b biofilm inhibition by IgG fractions purified from pre and post-immune sera of rabbits immunized against recombinant gene products of SE1501 and SE2232 in a 96-well polystyrene plate. Bacteria were suspended in 0.9% NaCl containing the indicated concentrations of polyclonal antibodies and incubated for 2 h at 4° C. and then overnight at 37° C. Formation of the biofilm was measured with safranin O. Percent inhibition of biofilm accumulation was determined from the formula (A₄₉₂, positive−A₄₉₂, antibody)/(A₄₉₂, positive−A₄₉₂, negative)×100%. (Sun, D. M. et al., 2005, Clin. Diagn. Lab. Immunol. 12:93-100).

FIG. 5. Sequence alignment (Clustal W, version 1.82) of SE2232 and highly homologous Staphylococcus proteins. SepiRP62A is a cell wall surface anchor family protein of S. epidermis RP62A; ShemJCSC1435 is the hypothetical protein SH0356 of S. hemolyticus JCSC1435; SaurCOL is the hypothetical LPxTG protein SA04HSC_{—}02982 from S. aureus ssp. aureus COL; SaurMW2 is the hypothetical protein MW2567 of S. aureus ssp. aureus MW2; SaurMu50 is the hypothetical protein SAV2646 from S. aureus ssp. aureus Mu50; SaurRF122 is a protein surface anchored protein of S. aureus RF122; SaurMRSA252 is a putative protein of S. aureus ssp. aureus MRSA252; Ssapr is the hypothetical protein SSPP105 from S. saprophyticus ssp. saprophyticus ATCC 15305.

FIG. 6. Comparison of biofilm inhibition in two strong biofilm-forming Staphylococcus epidermidis strains 10b and 1457 by rabbit polyclonal anti-SE2232 (SesC) IgGs and IgGs purified from pre-immune rabbit serum. A dose-dependent effect of biofilm inhibition is visible against biofilms of both strains.

FIG. 7. Gene expression of SE2232 (sesC) in sessile bacteria in a rat model for in vivo catheter infection during the 336 hours after implantation. A total of 192 polyurethane catheter segments were implanted subcutaneously in the rat model, and these segments were explanted at 11 different time-points. Data for each in vivo time point was obtained from sixteen independent measurements generated in two independent experiments. All catheter fragments from the same animal were used for a single time point. In each experiment, baseline expression levels in sessile bacteria before implantation were determined (time zero; n=16). The expression level at any time point is represented as the log₁₀ cDNA/gDNA ratio (Y axis) versus time (in hours) on the X axis. The error bars indicate standard errors.

### DETAILED DESCRIPTION OF THE INVENTION

“LPxTG motif” as used herein refers to the occurrence of a sequence of 5 amino acids in a microbial protein, where said 5 amino acids are: Leu-Pro-Xaa-Thr-Gly, in which Xaa indicates any natural occurring amino acid.

“Identity” or “sequence identity”, “similarity” and “sequence similarity”, “homology” or “sequence homology” all refer to the degree of correspondence of one stretch of nucleotides or amino acids with another sequence of nucleotides or amino acids. Highly homologous in this sense means that an amino acid sequence has a sequence identity of more than 50%, preferably more than 70%, more preferably more than 80%, more preferably more than 90%, even more preferably more than 95% and most preferably more than 98% with the above mentioned sequence.

“Identity” and “similarity” can be readily calculated by known methods (Computational Molecular Biology, Lesk, A. M., ed., Oxford University Press, New York, 1988; Biocomputing: Informatics and Genome Projects, Smith, D. W., ed., Academic Press, New York, 1993). While there exist a number of methods to measure identity and similarity between two sequences, both terms are well known to skilled artisans. Methods commonly employed to determine identity or similarity between sequences include, but are not limited to those disclosed in Carillo, H., and Lipman, D., SIAM J. Applied Math., 48:1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, GCG program package (Devereux et al., Nucleic Acids Research 12(1): 387, 1984), BLASTP, BLASTN, and FASTA (Atschul et al., J. Molec. Biol. 215: 403-410, 1990). The BLAST X program is publicly available from NCBI and other sources (BLAST Manual, Altschul et al., NCBI NLM NIH Bethesda, Md. 20894; Altschul et al., J. Mol. Biol. 215: 403-410, 1990). BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, 1993 Comput. Chem. 17:149-163) and XNU (Claverie and States, 1993 Comput. Chem. 17:191-201) low-complexity filters can be employed alone or in combination. The BLAST program used in the present invention for determining homology between proteins is BLASTP 2.2.16 (Altschul, S. F. et al. 1997, Nucleic Acids Res. 25:3389-3402; Schäffer, A. A. et al., 2001, Nucleic Acids Res. 29:2994-3005)

As used herein, ‘sequence identity’ or ‘identity’ or ‘homology’ in the context of two protein sequences (or nucleotide sequences) includes reference to the residues in the two sequences which are the same when aligned for maximum correspondence over a specified comparison window. When percentage of sequence identity is used in reference to proteins it is recognised that residue positions which are not identical often differ by conservative amino acid substitutions, where amino acids are substituted for other amino acid residues with similar chemical properties (e.g. charge or hydrophobicity) and therefore do not change the functional properties of the molecule. Where sequences differ in conservative substitutions, the percentage sequence identity may be adjusted upwards to correct for the conservative nature of the substitutions. Sequences, which differ by such conservative substitutions are said to have ‘sequence similarity’ or ‘similarity’. Means for making these adjustments are well known to persons skilled in the art. Typically this involves scoring a conservative substitution as a partial rather than a full mismatch, thereby increasing the percentage sequence identity. Thus, for example, where an identical amino acid is given a score of 1 and a non-conservative substitution is give a score of zero, a conservative substitution is given a score between 0 and 1. The scoring of conservative substitutions is calculated, e.g. according to the algorithm of Meyers and Miller (Computer Applic. Biol. Sci. 4:11-17, 1988).

“CONS” bacteria or “coagulase-negative” bacteria are bacteria of the genus Staphylococcus except for S. aureus and S. intermedius. However, all bacteria of the genus Staphylococcus are capable of biofilm formation.

By “biofilm” is meant the mass of microorganisms attached to a surface, such as a surface of a medical device, and the associated extracellular substances produced by one or more of the attached microorganisms. The extracellular substances are typically polymeric substances and commonly comprise a matrix of complex polysaccharides, proteinaceous substances and glycopeptides. This matrix or biofilm is also commonly referred to as “glycocalyx.” Biofilms can not only occur on insertable medical devices (“foreign bodies”), but also appear as a consequence of a bacterial infection on structures of the human or animal body itself. Clinically important are biofilm formation by S. aureus connected with otitis media and vaginal infections.

Biofilm formation on the surfaces of implantable or insertable medical devices (“foreign bodies”) adapted for long-term implantation, e.g., from about 30 days to 12 months or longer, can result in eventual encrustation and failure of the device. Further, the proliferation of microbes within the biofilm can lead to localized infections as well as difficulty to treat systemic infections. The extracellular substances that comprise the biofilm matrix can act as a barrier that protects and isolates the microorganisms housed in the biofilm from normal immunological defence mechanisms, such as antibodies and phagocytes, as well as from antimicrobial agents including surfactants, biocides and antibiotics. The biofilm also facilitates the growth and proliferation of microbes housed within the biofilm.

The present invention substantially reduces the risk of biofilm accumulation such as on the surfaces of a medical device adapted for long term implantation, and the resultant likelihood of premature failure of the device due to encrustation and occlusion by such biofilm. In some preferred embodiments of the present invention, the medical device is intended to remain implanted for a relatively long period of from about 30 days to about 12 months or longer. However, it is understood that the device may be implanted for a period of 30 days or shorter as well. Especially important in this respect is the application of the invention in neonates, where catheter-mediated CoNS infections occur in the first week after birth.

As indicated above, Staphylococcus spp., especially S. epidermis, S. aureus, S. saprophyticus and S. hemolyticus plays a major role in biofilm formation on foreign bodies, and essentially the cell wall associated (CWA) proteins play a major role during the early stages of Staphylococcus colonization and the later establishment of disease. One of the common factors of the known CWA proteins Aap, Bhp, SdrF and SdrG is that they are LPxTG motif containing proteins, indicating cell wall anchoring. In publicly available genomes of S. epidermidis strains RP62A and ATCC 12228, eleven and ten genes encoding LPxTG proteins have been identified respectively, of which eight genes, (SE2395 or sdrF, SE0331 or sdrG, SE0175 or aap, SE1429, SE2162, SE2232, SE0828 and SE1628) are present in both strains and five in only one of the two strains. Two hypothetical proteins, SE1500 and SE1501, are present in strain ATCC 12228 but not in RP62A, whereas the ORF's encoding SERP2392 (Bhp), SERP1482 and SERP1654 are only found in the S. epidermidis RP62A genome. Four genes encode the previously described proteins (Aap, Bhp, SdrF and SdrG), while the remaining ones have not been characterized yet.

We investigated the gene expression in planktonic and sessile bacteria of two hypothetical LPxTG proteins Lc. SE1501 and SE2232. The SE1501 gene encodes a hypothetical protein of 415 amino acids. The protein is similar to a few other known proteins including 1,4-alpha-glucan branching enzyme from Clostridium beijerincki and 2′,3′-cyclic-nucleotide 2′-phosphodiesterase from Clostridium perfringens (respectively 48% and 49% identity). The SE2232 gene (also designated as sesC in the literature, see Gill, S. R. et al., 2005, J. Bacteriol. 187:2426-2438) encodes a hypothetical protein of 676 amino acids (see FIG. 5). This protein exhibits some similarity to the much larger AAS surface protein of Staphylococcus saprophyticus (Hell, W. et al., 1998, Mol. Microbiol. 29:871-881) and staphylococcal fibronectin binding proteins such as AtlC from Staphylococcus caprae (Allignet, J. et al., 2001, Infect. Immun. 69:712-718) with respectively 43% and 44% identity. Highest homology, however, is found to some putative or hypothetical proteins from S. aureus, S. hemolyticus and S. saprophyticus, as is shown in FIG. 5. The e-values obtained from a BLASTP comparison of SE2232 with the various proteins listed were 2e⁻⁷⁸ for S. hemolyticus JCSC1435, 5e⁻⁷⁷ for S. aureus COL, 8e⁻⁷⁷ for S. aureus MW2 and S. aureus Mu50, 3e⁻⁷⁵ for S. aureus RF122 and MRSA252 and 3e⁻⁵³ for S. saprophyticus.

As is shown in the experimental part, it is surprising that inhibiting the SE2232 protein (through rabbit polyclonal antibody serum) was able to significantly prevent biofilm formation, whereas inhibition of SE1501 did not yield significant results. The invention thus provides a method to prevent biofilm formation on a surface by coating said surface with antibodies against SE2232 or antibodies generated against any of the proteins listed in FIG. 5.

As used herein, the terms “antibody” and “antibodies” refer to monoclonal antibodies, multispecific antibodies (e.g., bi-specific), human antibodies, humanized antibodies, camelised antibodies, chimeric antibodies, single-chain Fvs (scFv), single chain antibodies, synthetic antibodies, single domain antibodies, Fab fragments, F(ab) fragments, disulfide-linked Fvs (sdFv), and anti-idiotypic (anti-Id) antibodies, and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

Antibodies of the invention include, but are not limited to, monoclonal antibodies, multispecific antibodies, synthetic antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies of the present invention include immunoglobulin molecules and immunologically active portions of immunoglobulin molecules, i.e., molecules that contain an antigen binding site that immunospecifically binds to an SE2232 antigen or an antigen against any of the proteins listed in FIG. 5. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The antibodies of the invention may be from any animal origin including birds and mammals (e.g., human, murine, donkey, sheep, rabbit, goat, guinea pig, camel, horse, or chicken). As used herein, “human” antibodies include antibodies having the amino acid sequence of a human immunoglobulin and include antibodies isolated from human immunoglobulin libraries (including, but not limited to, synthetic libraries of immunoglobulin sequences homologous to human immunoglobulin sequences) or from mice that express antibodies from human genes.

In certain embodiments, high potency antibodies can be used in the methods of the invention. For example, high potency antibodies can be produced by genetically engineering appropriate antibody gene sequences and expressing the antibody sequences in a suitable host. The antibodies produced can be screened to identify antibodies with, e.g., high kₒₙ values in a BIAcore assay.

In certain embodiments, an antibody to be used with the methods of the present invention or fragment thereof has an affinity constant or Kₐ (kₒₙ/k_{off}) of, at least 10² M⁻¹, at least 5×10² M⁻¹, at least 10³ M⁻¹, at least 5×10³ M⁻¹, at least 10⁴M⁻¹, at least 5×10⁴M⁻¹, at least 10⁵ M⁻¹, at least 5×10⁵M⁻¹, at least 10⁶ M⁻¹, at least 5×10⁶ M⁻¹, at least 10⁷M⁻¹, at least 5×10⁷M⁻¹, at least 10⁸ at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ at least 10¹⁵ M⁻¹, or at least 5×10¹⁵ M⁻¹. In yet another embodiment, an antibody to be used with the methods of the invention or fragment thereof has a dissociation constant or K_{d} (k_{off}/kₒₙ) of less than 10⁻² M, less than 5×10⁻² M, less than 10⁻³ M, less than 5×10⁻³ M, less than 10⁻⁴ M, less than 5×10⁻⁴ M, less than 10⁻⁵ M, less than 5×10⁻⁵ M, less than 10⁻⁶ M, less than 5×10⁻⁶ M, less than 10⁻⁷ M, less than 5×10⁻⁷M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹M, less than 10⁻¹² M, less than 5×10⁻¹² M, less than 10⁻¹³M, less than 5×10⁻¹³ M, less than 10⁻¹⁴ M, less than 5×10⁻¹⁴M, less than 10⁻¹⁵ M, or less than 5×10⁻¹⁵ M.

In certain embodiments, an antibody to be used with the methods of the invention or fragment thereof that has a median effective concentration (EC₅₀) of less than 0.01 nM, less than 0.025 nM, less than 0.05 nM, less than 0.1 nM, less than 0.25 nM, less than 0.5 nM, less than 0.75 nM, less than 1 nM, less than 1.25 nM, less than 1.5 nM, less than 1.75 nM, or less than 2 nM, in an in vitro biofilm inhibition assay (see experimental part). The median effective concentration is the concentration of antibody or antibody fragments that inhibits 50% of the biofilm formation in said biofilm inhibition assay. In a preferred embodiment, an antibody to be used with the methods of the invention or fragment thereof has an EC₅₀ of less than 0.01 nM, less than 0.025 nM, less than 0.05 nM, less than 0.1 nM, less than 0.25 nM, less than 0.5 nM, less than 0.75 nM, less than 1 nM, less than 1.25 nM, less than 1.5 nM, less than 1.75 nM, or less than 2 nM, in biofilm inhibition assay.

The antibodies to be used with the methods of the invention include derivatives that are modified, i.e, by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, synthesis in the presence of tunicamycin, etc. Additionally, the derivative may contain one or more non-classical amino acids.

The present invention also provides antibodies of the invention or fragments thereof that comprise a framework region known to those of skill in the art. In certain embodiments, one or more framework regions, preferably, all of the framework regions, of an antibody to be used in the methods of the invention or fragment thereof are human. In certain other embodiments of the invention, the fragment region of an antibody of the invention or fragment thereof is humanized. In certain embodiments, the antibody to be used with the methods of the invention is a synthetic antibody, a monoclonal antibody, an intrabody, a chimeric antibody, a human antibody, a humanized chimeric antibody, a humanized antibody, a glycosylated antibody, a multispecific antibody, a human antibody, a single-chain antibody, or a bispecific antibody. In certain embodiments of the invention, the antibodies to be used with the invention have half-lives in a mammal, preferably a human, of greater than 12 hours, greater than 1 day, greater than 3 days, greater than 6 days, greater than 10 days, greater than 15 days, greater than 20 days, greater than 25 days, greater than 30 days, greater than 35 days, greater than 40 days, greater than 45 days, greater than 2 months, greater than 3 months, greater than 4 months, or greater than 5 months. Antibodies or antigen-binding fragments thereof having increased in vivo half-lives can be generated by techniques known to those of skill in the art. For example, antibodies or antigen-binding fragments thereof with increased in vivo half-lives can be generated by modifying (e.g., substituting, deleting or adding) amino acid residues identified as involved in the interaction between the Fc domain and the FcRn receptor (see, e.g., PCT Publication No. WO 97/34631 and U.S. patent application Ser. No. 10/020,354, entitled “Molecules with Extended Half-Lives, Compositions and Uses Thereof”, filed Dec. 12, 2001, by Johnson et al., which are incorporated herein by reference in their entireties). Such antibodies or antigen-binding fragments thereof can be tested for binding activity to SE2232 antigens or any of the other proteins listed in FIG. 5, as well as for in vivo efficacy using methods known to those skilled in the art, for example, by assays as described herein.

Further, antibodies or antigen-binding fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography. PEG-derivatizated antibodies or antigen-binding fragments thereof can be tested for binding activity to SE2232 antigens as well as for in vivo efficacy using methods known to those skilled in the art, for example, by assays as described herein.

In certain embodiments, the antibodies to be used with the methods of the invention are fusion proteins comprising an antibody or fragment thereof that immunospecifically binds to a SE2232 antigen and a heterologous polypeptide. Preferably, the heterologous polypeptide that the antibody or antibody fragment is fused to is useful for coating of foreign bodies or devices inserted into the human or mammalian body.

In certain embodiments, antibodies to be used with the methods of the invention are single-chain antibodies (sFv). The design and construction of a single-chain antibody is described in Marasco et al, 1993, Proc Natl Acad Sci 90:7889-7893, which is incorporated herein by reference in its entirety. The sFv typically comprises a single peptide with the sequence V_{H}-linker-V_{L} or V_{L}-linker-V_{H}. The linker is chosen to permit the heavy chain and light chain to bind together in their proper conformational orientation (see for example, Huston, et al., 1991, Methods in Enzym. 203:46-121, which is incorporated herein by reference). In a further embodiment, the linker can span the distance between its points of fusion to each of the variable domains (e.g., 3.5 nm) to minimize distortion of the native Fv conformation. In such an embodiment, the linker is a polypeptide of at least 5 amino acid residues, at least 10 amino acid residues, at least 15 amino acid residues, or greater. In a further embodiment, the linker should not cause a steric interference with the V_{H} and V_{L} domains of the combining site. In such an embodiment, the linker is 35 amino acids or less, 30 amino acids or less, or 25 amino acids or less. Thus, in a most preferred embodiment, the linker is between 15-25 amino acid residues in length. In a further embodiment, the linker is hydrophilic and sufficiently flexible such that the V_{H} and V_{L} domains can adopt the conformation necessary to detect antigen. Examples of linkers include, but are not limited to, those sequences disclosed in Table 1.

TABLE 1
Sequence

(Gly Gly Gly Gly Ser)₃
Glu Ser Gly Arg Ser Gly Gly Gly Gly Ser Gly Gly Gly Gly Ser
Glu Gly Lys Ser Ser Gly Ser Gly Ser Glu Ser Lys Ser Thr
Glu Gly Lys Ser Ser Gly Ser Gly Ser Glu Ser Lys Ser Thr Gln
Glu Gly Lys Ser Ser Gly Ser Gly Ser Glu Ser Lys Val Asp
Gly Ser Thr Ser Gly Ser Gly Lys Ser Ser Glu Gly Lys Gly
Lys Glu Ser Gly Ser Val Ser Ser Glu Gln Leu Ala Gln Phe Arg Ser Leu Asp
Glu Ser Gly Ser Val Ser Ser Glu Glu Leu Ala Phe Arg Ser Leu Asp

The antibodies to be used with the methods of the invention or fragments thereof can be produced by any method known in the art for the synthesis of antibodies, in particular, by chemical synthesis or preferably, by recombinant expression techniques.

Polyclonal antibodies to an SE2232 antigen or an antigen from the proteins listed in FIG. 5 can be produced by various procedures well known in the art. For example, an SE2232 antigen can be administered to various host animals including, but not limited to, rabbits, mice, rats, etc. to induce the production of sera containing polyclonal antibodies specific for the SE2232 antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, vitamin E and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in; Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art. Briefly, mice can be immunized with an SE2232 antigen and once an immune response is detected, e.g., antibodies specific for the SE2232 antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

In certain embodiments, a method of generating monoclonal antibodies comprises culturing a hybridoma cell secreting an antibody of the invention wherein, preferably, the hybridoma is generated by fusing splenocytes isolated from a mouse immunized with an SE2232 antigen with myeloma cells and then screening the hybridomas resulting from the fusion for hybridoma clones that secrete an antibody able to bind an SE2232 antigen.

Antibody fragments which recognize specific SE2232 epitopes or epitopes of any of the proteins listed in FIG. 5, may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies to be used with the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VII and VL domains are usually recombinantly fused to either the phage gene III or gene VIII. Phage expressing an antigen binding domain that binds to a SE2232 antigen can be selected or identified with antigen, e.g., using labelled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT application No. PCT/GB91/O1 134; PCT publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties). To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VII or VL domains comprise an EF-1α promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for coating foreign bodies which are introduced into human subjects. Human antibodies can be made by a variety of methods known in the art including phage display methods described above using antibody libraries derived from human immunoglobulin sequences or synthetic sequences homologous to human immunoglobulin sequences. See also U.S. Pat. Nos. 4,444,887 and 4,716,111; and PCT publications WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then being bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entireties. In addition, companies such as Medarex, Inc. (Princeton, N.J.), Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules such as antibodies having a variable region derived from a non-human (e.g., murine) antibody and a human immunoglobulin constant region. Methods for producing chimeric antibodies are known in the art. See e.g., Morrison, 1985, Science 229:1202; Oi et al., 1986, BioTechniques 4:214; Gullies et al., 1989, J. Immunol. Methods 125:191-202; and U.S. Pat. Nos. 5,807,715, 4,816,567, and 4,816,397, which are incorporated herein by reference in their entireties. Chimeric antibodies comprising one or more CDRs from human species and framework regions from a non-human immunoglobulin molecule can be produced using a variety of techniques known in the art including, for example, CDR-grafting (EP 239,400; PCT publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(415):489-498; Studnicka et al., 1994, Protein Engineering 7(6):805-814; and Roguska et al., 1994, PNAS 91:969-973), and chain shuffling (U.S. Pat. No. 5,565,332). Often, framework residues in the framework regions will be substituted with the corresponding residue from the CDR donor antibody to alter, preferably improve, antigen binding. These framework substitutions are identified by methods well known in the art, e.g., by modelling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.)

In certain embodiments, a fragment of a protein of SE2232 is used as an immunogen for the generation of antibodies to be used with the methods of the invention. A fragment of a protein of SE2232 to be used as an immunogen can be at least 10, 20, 30, 40, 50, 75, 100, 250, 500, 750, or at least 1000 amino acids in length. In certain embodiments a synthetic peptide of a protein of SE2232 is used as an immunogen.

Polynucleotides encoding antibodies to be used with the invention may be obtained, and the nucleotide sequence of the polynucleotides determined, by any method known in the art. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR. Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence coding for the antibody is determined, said nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, a nucleotide coding for one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide generated by the combination of the framework regions and CDRs encodes an antibody that specifically binds to an SE2232 antigen and/or an antigen of one of the proteins listed in FIG. 5. In certain embodiments, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

Recombinant expression of an antibody to be used with the methods of the invention, derivative or analogue thereof, (e.g., a heavy or light chain of an antibody of the invention or a portion thereof or a single chain antibody of the invention), requires construction of an expression vector containing a polynucleotide that encodes the antibody. Once a polynucleotide encoding an antibody molecule or a heavy or light chain of an antibody, or portion thereof (preferably, but not necessarily, containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy, the entire light chain, or both the entire heavy and light chains. The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below. A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies or antigen-binding fragments thereof which immunospecifically bind to one or more SE2232 antigens and/or antigens of the proteins listed in FIG. 5 is regulated by a constitutive promoter, inducible promoter or tissue specific promoter.

In bacterial systems, a number of expression vectors may be advantageously selected depending upon the use intended for the antibody molecule being expressed. For example, when a large quantity of such a protein is to be produced, for the generation of antibody molecules for coating of devices, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example, the polyhedrin gene) of the virus and placed under control of an AcNPV promoter (for example, the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk−, hgprt− or aprt− cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule to be used with the methods of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

In certain embodiments, the antibodies to be used with the methods of the invention or fragments thereof are recombinantly fused or chemically conjugated (including both covalently and non-covalently conjugations) to a heterologous polypeptide (or portion thereof, preferably at least 10, at least 20, at least 30, at least 40, at least 50, at least 60, at least 70, at least 80, at least 90 or at least 100 amino acids of the polypeptide) to generate fusion proteins. The fusion does not necessarily need to be direct, but may occur through linker sequences. In certain embodiments, the anti-SE2232-antigen antibody is an antibody conjugate.

Additional fusion proteins of the antibodies to be used with the methods of the invention or fragments thereof may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or antigen-binding fragments thereof with higher affinities and lower dissociation rates). See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998) (each of these patents and publications are hereby incorporated by reference in its entirety). In one embodiment, antibodies or antigen-binding fragments thereof, or the encoded antibodies or antigen-binding fragments thereof, may be altered by being subjected to random mutagenesis by error-prone PCR, random nucleotide insertion or other methods prior to recombination. In another embodiment, one or more portions of a polynucleotide encoding an antibody or antibody fragment, which portions immunospecifically bind to an SE2232 antigen and/or an antigen of one or more of the proteins listed in FIG. 5 may be recombined with one or more components, motifs, sections, parts, domains, fragments, etc. of one or more heterologous molecules.

Moreover, the antibodies to be used with the methods of the present invention or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin“HA” tag, which corresponds to an epitope derived from the influenza hemagglutinin protein (Wilson et al., 1984, Cell 37:767) and the “flag” tag.

An antibody or fragment thereof may be conjugated to a therapeutic moiety such as, but not limited to, a antibacterial compound or a therapeutic agent. Antibiotics to be used comprise bacteriocins, such as nisin and pediocin; antibiotics, such as penicillin, erythromycin, ampicillin, isoniazid, tetracycline, sulphonamides and chloramphenicol; vegetable toxins, such as defensins, lectins, and anti-fungal proteins; H₂O₂-producing enzymes such as oxidases; sodium diacetate; sodium nitrite; lysozyms and antimicrobial substances from spices. Further, an antibody to be used with the methods of the invention or fragment thereof may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, but are not limited to, a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Iminunol., 6:1567-1574), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Techniques for conjugating such therapeutic moieties to antibodies are well known, see, e.g., Hellstrom et al., “Antibodies For Drug Delivery”, in Controlled Drug Delivery (2nd Ed.), Robinson et al. (eds.), pp. 623-53 (Marcel Dekker, Inc. 1987) and Thorpe et al., 1982, Immunol. Rev. 62:119-58.

One embodiment of the invention provides in medical devices or foreign bodies that are coated with antibodies, as discussed above, that immunospecifically bind to SE2232 and/or one or more of the proteins listed in FIG. 5 or homologues thereof. For this invention homologues of the proteins listed in FIG. 5 are defined as proteins which have an e-value of 1e⁻⁵⁰ or lower, preferably 1e⁻⁷⁵ or lower, in a BLASTP comparison with one or more of the proteins listed in FIG. 5.

Medical devices or polymeric biomaterials to be coated with the antibodies described herein include, but are not limited to, staples, sutures, replacement heart valves, cardiac assist devices, hard and soft contact lenses, intraocular lens implants (anterior chamber or posterior chamber), other implants such as corneal inlays, kerato-prostheses, vascular stents, epikeratophalia devices, glaucoma shunts, retinal staples, scleral buckles, dental prostheses, thyroplastic devices, laryngoplastic devices, vascular grafts, cerebrovascular shunts, soft and hard tissue prostheses including, but not limited to, pumps, electrical devices including stimulators and recorders, auditory prostheses, pacemakers, artificial larynx, dental implants, mammary implants, penile implants, cranio/facial tendons, artificial joints, tendons, ligaments, menisci, and disks, artificial bones, artificial organs including artificial pancreas, artificial hearts, artificial limbs, and heart valves; stents, wires, guide wires, intravenous and central venous catheters, laser and balloon angioplasty devices, vascular and heart devices (tubes, catheters, balloons), ventricular assists, blood dialysis components, blood oxygenators, urethral/ureteral/urinary devices (Foley catheters, stents, tubes and balloons), airway catheters (endotracheal and tracheostomy tubes and cliffs), enteral feeding tubes (including nasogastric, intragastric and jejunal tubes), wound drainage tubes, tubes used to drain the body cavities such as the pleural, peritoneal, cranial, and pericardial cavities, blood bags, test tubes, blood collection tubes, vacutainers, syringes, needles, pipettes, pipette tips, and blood tubing. The medical devices may be formed of any suitable metallic materials or non-metallic materials known to persons skilled in the art. Examples of metallic materials include, but are not limited to, tivanium, titanium, and stainless steel, and derivatives or combinations thereof. Examples of non-metallic materials include, but are not limited to, thermoplastic or polymeric materials such as rubber, plastic, polyesters, polyethylene, polyurethane, silicone, Gortex (polytetrafluoroethylene), Dacron™, (polyethylene tetraphthalate), Teflon (polytetrafluoroethylene), latex, elastomers and Dacron™ sealed with gelatin, collagen or albumin, and derivatives or combinations thereof.

Stents include biliary, urethral, ureteral, tracheal, coronary, gastrointestinal and oesophageal stents. The stents may be of any shape or configuration. The stents may comprise a hollow tubular structure which is particularly useful in providing flow or drainage through lumens. Stents may also be coiled or patterned as a braided or woven open network of fibres or filaments or, for example, as an interconnecting open network of articulable segments. Such stent designs may be more particularly suitable for maintaining the patency of a body lumen such as a coronary artery. Thus, stents adapted primarily to provide drainage, in contrast to stents adapted primarily to support a body lumen, will preferably have a continuous wall structure in contrast to an open network wall structure. The stent can be made of any material useful for such purpose including metallic and nonmetallic materials as well as shape memory materials. Useful metallic materials include, but are not limited to, shape memory alloys such as Nitinol™, and other metallic materials including, but not limited to, stainless steel, tantalum, nickel-chrome, or cobalt-chromium, i.e., Elgiloy™. The stent can be made from a single strand or multiple strands of any such material and may be self-expanding.

Stent covers are also a preferred medical device of the present invention. For example, a stent cover may comprise a thin wall tubular or sheath-like structure adapted to be placed over a stent comprising an open mesh stent of knitted, woven or braided design.

The coated devices can be used in both humans and mammals such as pet animals, livestock animals, zoo animals and the like. Preferred non-human mammals are cat, dog, swine and cattle.

It will be understood by those skilled in the art that the term “coated” or “coating”, as used herein, means to apply the antibody or binding fragment of the invention to a surface of the device, preferably an outer surface that would be exposed to coagulase-negative staphylococcal infection. The surface of the device does not need to be entirely covered by the antibody or fragment. Coating of antibodies onto substrates is a technique commonly applied in the field of diagnostic assays and biosensors. Any coating method used in these fields may be applied according to the present invention. Preferably, and most simple, coating is provided by immersion of the surface to be coated in a solution comprising the antibody, or, alternatively, by spreading a solution comprising the antibodies onto the surface to be coated.

Natural biofilm formation (i.e. formation of a biofilm which is not deposited onto a “foreign body”) occurs sometimes during infection with micro-organisms, especially S. aureus, especially in otitis media (middle ear infection), vaginal infections and mastitis. In order to prevent or treat such natural biofilm formation, also the antibodies of the invention can be used, especially the antibodies developed against the S. aureus proteins listed in FIG. 5. However, as alternative to the administration of an antibody to the patient, it is also possible to vaccinate the patient with a subunit vaccine, comprising the protein or an immunogenically active (i.e. antigenic) part thereof.

The route of administration for any one of the embodiments of the vaccine of the present invention includes, but is not limited to, oronasal, intramuscular, intraperitoneal, intradermal, subcutaneous, intravenous, intraarterial, intraocular, intravaginal and oral as well as transdermal or by inhalation or suppository. The preferred routes of administration include oronasal, intramuscular, intraperitoneal, intradermal, intravaginal and subcutaneous injection. The vaccine can be administered by any means that includes, but is not limited to, syringes, nebulizers, misters, needleless injection devices, or microprojectile bombardment gene guns (Biolistic bombardment).

The vaccine for any one of the embodiments of the present invention is formulated in a pharmaceutically accepted carrier according to the mode of administration to be used. One skilled in the art can readily formulate a vaccine that comprises a protein listed in FIG. 5, a homologous protein thereof, or an immunogenic fragment thereof. In cases where intramuscular injection is preferred, an isotonic formulation is preferred. Generally, additives for isotonicity can include sodium chloride, dextrose, mannitol, sorbitol, and lactose. In particular cases, isotonic solutions such as phosphate buffered saline are preferred. The formulations can further provide stabilizers such as gelatin and albumin. In some embodiments, a vaso-constrictive agent is added to the formulation. The pharmaceutical preparations according to the present invention are provided sterile and pyrogen-free. However, it is well known by those skilled in the art that the preferred formulations for the pharmaceutically accepted carrier which comprise the vaccines of the present invention are those pharmaceutical carriers approved in the regulations promulgated by the United States Department of Agriculture, or equivalent government agency in a foreign country such as Canada or Mexico or any one of the European nations. Therefore, the pharmaceutically accepted carrier for commercial production of the vaccine of the present invention is a carrier that is already approved or will be approved by the appropriate government agency. The vaccine can further be mixed with an adjuvant that is pharmaceutically acceptable. In certain formulations of the vaccine of the present invention, the vaccine is combined with other vaccines to produce a polyvalent vaccine product that can protect subjects not only with respect to biofilm formation, but which will also give protection against the micro-organisms, especially S. aureus, which cause the infection.

Administration is preferably by a single vaccination that produces a full, broad immunogenic response. In another embodiment of the present invention, the subject is treated with a series of vaccinations to produce a full, broad immune response. When the vaccinations are provided in a series, the vaccinations can be provided between about one day to four weeks or longer apart.

The vaccine compositions optionally may include vaccine-compatible pharmaceutically acceptable (i.e., sterile and non-toxic) liquid, semisolid, or solid diluents that serve as pharmaceutical vehicles, excipients, or media. Diluents can include water, saline, dextrose, ethanol, glycerol, and the like. Isotonic agents can include sodium chloride, dextrose, mannitol, sorbitol, and lactose, among others. Stabilizers include albumin, among others. Any adjuvant known in the art may be used in the vaccine composition, including oil-based adjuvants such as Freund's Complete Adjuvant and Freund's Incomplete Adjuvant, mycolate-based adjuvants (e.g., trehalose dimycolate), bacterial lipopolysaccharide (LPS), peptidoglycans (i.e., mureins, mucopeptides, or glycoproteins such as N-Opaca, muramyl dipeptide [MDP], or MDP analogs), proteoglycans (e.g., extracted from Klebsiella pneumoniae), streptococcal preparations (e.g., OK432), Biostim™ (e.g., 01K2), the “Iscoms” of EP 109 942, EP 180 564 and EP 231 039, aluminum hydroxide, saponin, DEAE-dextran, neutral oils (such as miglyol), vegetable oils (such as arachis oil), liposomes, Pluronic® polyols. Adjuvants include, but are not limited to, the RIBI adjuvant system (Bibi Inc.), alum, aluminum hydroxide gel, cholesterol, oil-in water emulsions, water-in-oil emulsions such as, e.g., Freund's complete and incomplete adjuvants, Block co-polymer (CytRx, Atlanta Ga.), SAF-M (Chiron, Emeryville Calif.), AMPHIGEN® adjuvant, saponin, Quil A, QS-21 (Cambridge Biotech Inc., Cambridge Mass.), GPI-0100 (Galenica Pharmaceuticals, Inc., Birmingham, Ala.) or other saponin fractions, monophosphoryl lipid A, Avridine lipid-amine adjuvant, heat-labile enterotoxin from E. coli (recombinant or otherwise), cholera toxin, or muramyl dipeptide, among many others. The immunogenic compositions can further include one or more other immunomodulatory agents such as, e.g., interleukins, interferons, or other cytokines. The immunogenic compositions can also include gentamicin and merthiolate. While the amounts and concentrations of adjuvants and additives useful in the context of the present invention can readily be determined by the skilled artisan, the present invention contemplates compositions comprising from about 50 μg to about 2000 μg of adjuvant and preferably about 500 μg/2 ml dose of the vaccine composition. In another preferred embodiment, the present invention contemplates vaccine compositions comprising from about 1 μg/ml to about 60 μg/ml of antibiotic, and more preferably less than about 30 μg/ml of antibiotic.

The immunogenic compositions of the present invention can be made in various forms depending upon the route of administration. For example, the immunogenic compositions can be made in the form of sterile aqueous solutions or dispersions suitable for injectable use, or made in lyophilized forms using freeze-drying techniques. Lyophilized immunogenic compositions are typically maintained at about 4° C., and can be reconstituted in a stabilizing solution, e.g., saline or/and HEPES, with or without adjuvant.

In addition, the immunogenic and vaccine compositions of the present invention can include one or more pharmaceutically-acceptable carriers. As used herein, “a pharmaceutically-acceptable carrier” includes any and all solvents, dispersion media, coatings, adjuvants, stabilizing agents, diluents, preservatives, antibacterial and antifungal agents, isotonic agents, adsorption delaying agents, and the like. The carrier(s) must be “acceptable” in the sense of being compatible with the components of the invention and not deleterious to the subject to be immunized. Typically, the carriers will be will be sterile and pyrogen-free.

The vaccines of the present invention, which are amongs other thought to inhibit biofilm formation, can be co-administered with any antibiotic treatment. It is envisaged that by preventing the formation of a biofilm, the bacteria are more accessible and thus more prone to antibiotic treatment.

### EXAMPLES

### Example 1

### Expression of SE2232 and SE1501 In Vitro and In Vivo

### Bacteria Used.

For all experiments, a previously well-characterized biofilm-producing S. epidermidis strain, strain 10b, was used. S. epidermidis 10b was isolated from a patient with a proven catheter-related bloodstream infection (31, 32, 33, 34). It is homogeneously oxacillin resistant and has a 100% infection rate in the rat model used (34).

### Gene Identification.

The sequences of the genes studied were retrieved from the National Center for Biotechnology Information (NCBI) GenBank (Table 1). On the basis of the sequences, primers were designed with Primer Express 2.0 software (Applied Biosystems Division of Perkin-Elmer) and used to amplify part of the corresponding genes in S. epidermidis 10b. The following thermal cycling conditions were used: 5 min at 95° C., followed by 25 repeats of 30 s at 95° C., 30 s at related annealing temperatures (Table 1) and 1 min at 72° C. followed by 7 min at 72° C. and holding at 4° C. PCR was performed on a GeneAmp PCR System 9700 (Perkin-Elmer Applied Biosystems, Foster City, Calif.). All primers and probes were provided by Eurogentec (Seraing, Belgium).

TABLE 1
sequences of primers (5′→3′), annealing temperature, fragment length and
number of copies of gene per μL plasmid solution.

| | | | | | Number of |
|---|---|---|---|---|---|
| | | | | Fragment | gene copies |
| | | | Annealing | Length | per μl of |
| | | | Temperature | (base | plasmid |
| Genes | Forward primer | Reverse primer | ° C.) | pair) | solution |
| SE1501 | CCAATTACTAGTATT | CTACACTGTTAGA | 52 | 355 | 1.93 × 10¹⁰ |
| | AAATTCAG | CGTGAG- | | | |
| SE2232 | GTTGATAACCGTCAA | CATGTTGATCTTTT | 62 | 388 | 1.51 × 10¹⁰ |
| | CAAGG | GAATCCC | | | |

### Plasmid Cloning and Quantification of Number of Copies of the Plasmid for Taqman Quantitative PCR.

All genes were cloned as described elsewhere (31, 35). Pure plasmid DNA was obtained and quantified with the GeneQuant RNA/DNA calculator (Amersham Pharmacia Biotech, Uppsala, Sweden).

### Gene Expression in In Vitro Bacteria.

The in vitro model has been described previously. Briefly, a frozen culture of S. epidermidis 10b was grown to the end-exponential growth phase, pelleted, and resuspended in 0.9% NaCl. Fragments (length; 7 mm) of a commercial polyurethane intravenous catheter (Arrow International, Reading, Pa.) were added to this bacterial suspension and placed in a water bath at 37° C. After different incubation periods, nucleic acids (RNA and DNA) were extracted instantaneously by an adaptation of the method of Cheung et al. (36) as previously described (31, 32, 33). Briefly, for bacteria in suspension (planktonic bacteria), an aliquot of a bacterial suspension was rapidly cooled on ice. The bacteria were pelleted, and the pellet was suspended in 500 μl of NAES buffer (50 mM sodium acetate [pH 5.1], 10 mM EDTA, 1% sodium dodecyl sulfate) and added to a FastRNA blue tube (Bio 101, Carlsbad, Calif.) with 500 μl acidified phenol-chloroform (5:1; pH 4.5; Ambion, Austin, Tex.) at room temperature. For the bacteria that adhered to a catheter segment, the colonized catheter fragment was washed with 1 ml of 0.9% NaCl and added directly to a FastRNA tube containing 500 μl of NAES buffer and 500 μl of acidified phenol-chloroform. The FastRNA tubes were shaken for 23 s at 6,000 rpm with a FastPrep instrument (FP 120; Bio 101, Savant, Holbrook, N.Y.). After shaking, the tubes were centrifuged, and 90% of the supernatant (450 μl) was precipitated with 520 μl of isopropyl alcohol and 35 μl of 3 M sodium acetate. The pellet was washed with 70% ethanol and resuspended in 150 μl of RNase-free water. Fifty microliters of this sample was diluted 1/10 and used for quantification of gDNA. The remaining 100 μl was purified with an Rneasy mini kit (Qiagen, Hilden, Germany) and treated with RNase-free DNase (Qiagen) on Rneasy columns according to the instructions of the manufacturer. The RNA was finally dissolved in 60 μl of RNase-free water. Reverse transcription of the RNA was performed with Moloney murine leukemia virus reverse transcriptase (Promega, Madison, Wis., USA) as previously described (31, 35, 37). Nucleic acid isolation was done at time point 0 min (n=6) (just before suspending the bacteria in 0.9% NaCl) and at time point 10 (n=12), 35 (n=12), 60 (n=12), 120 (n=12), and 180 (n=12) min, both from the bacteria adherent to the catheters and from planktonic bacteria. Data for all time points were generated in six independent measurements.

### Gene Expression in Sessile Bacteria During In Vivo Foreign Body Infection

We used a previously described model with some modifications in which first-generation descendants of inbred Fisher rats harbored under germfree conditions since 1965 were used (34). The rats used in our experiments were exposed to normal rat flora from birth and were labeled ex-germfree Fisher rats. In this model, catheter fragments were inoculated with a small amount of S. epidermidis 10b before implantation, which resulted in a 100% infection rate (34). The experimental conditions have been described in detail previously (31, 32, 33). Briefly, 7-mm catheter fragments were incubated for 20 min in a suspension of S. epidermidis in 0.9% NaCl as described above. After 20 min, the suspension with catheter fragments was placed on ice. Anesthesia of rats was induced with urethane and during the procedure the rats were kept anaesthetized with a combination of 20% urethane and 80% oxygen. The back of each rat was shaved over a large area, and the skin was generously disinfected with 0.5% chlorhexidine in 70% alcohol and allowed to dry for 2 min. A 10-mm skin incision was made at the base of the tail of each adult ex-germfree Fisher rat, and the subcutis was carefully dissected to create four subcutaneous tunnels. In each rat, eight catheter fragments were quickly inserted subcutaneously at least 2 cm from the incision; the distance between two fragments was at least 1 cm. For catheter explanation, animals were sacrificed by using 0.5% CO₂. The skin was disinfected as described above, and the naked catheter segments were gently removed from the subcutaneous tissue. All catheter fragments from one animal were used for one time point. In each experiment, baseline expression levels in sessile bacteria before implantation were determined (time zero; n=16). A total of 192 polyurethane catheter segments were implanted subcutaneously in the rat model, and these segments were explanted at 11 different time-points. The time-points used were 15 min (n=16), 1 h (n=16), 2 h (n=16), 4 h (n=16), 6 h (n=16), 12 h (n=16), 24 h (n=16), 2 days (n=16), 4 days (n=16), 7 days (n=16), and 14 days (n=16). Data for each in vivo time point were generated in sixteen independent measurements generated in two independent experiments. Nucleic acid isolation and cDNA synthesis were performed immediately after explanation as described above.

### Taqman Quantitative PCR.

Quantification of both cDNA and gDNA was done on the ABI Prism 7700 Sequence Detection System (PE Applied Biosystems), as described elsewhere (31, 35). In brief, quantitative PCR was done with 2 μL of cDNA, 12.5 μL of 2× Taqman PCR master mix (PE Applied Biosystems), 900 nmol/L of each primer, and 200 nmol/L probe in a final volume of 25 μL. Thermal cycling conditions were the following: 2 min at 50° C., followed by 10 min at 95° C., followed by 45 repeats of 15 s at 95° C. and 1 min at 60° C. Data collection was done during each annealing phase. During each run, a standard dilution of the plasmid with known quantity was included to permit gene quantification by means of the supplied software, according to the instructions of the manufacturer. In each run, a negative control (distilled water) was included. The primers and probes for the genes are summarized in Table 2.

### Statistical Analysis.

All statistical analyses were performed with Graphpadprism (Graphpad software version 4.2, San Diego, USA). Since the in vitro and in vivo cDNA/gDNA ratios were not normally distributed at any time point, all data were log₁₀ transformed in order to fulfil the requirements of normality.

TABLE 2
Taqman primers and probes (5′→3′) used to study the expression
of genes with putative roles in the pathogenesis of
Staphylococcus epidermidis foreign body infections.

| | | | Probe: labeled FAM-5′ | |
|---|---|---|---|---|
| Genes | PID | Forward primer | and 3′-TAMRA | Reverse primer |
| SE1501 | 27468419 | CCCTAAAGATGAAA | ACAATGTGACAAAACAGGG | TGCTATCCATTCGAAAC |
| | | CTACTGTTCATGAT | TGTACTAAAAGTAAACG | TTTCATTAT |
| SE2232 | 27469150 | AGCATCACCATCTAA | TAACAAAGAAGAATCTAGT | CCATCATTACTTTTATC |
| | | TAAAAACGAAA | ACGACAACAAATCAATCCG | GTCTTTACTATCAC |
| | | | A | |

For the in vitro data, two hypotheses were tested. A significant change in gene expression levels over time within one group (sessile or planktonic) was tested with a one-way analysis of variance (One-way ANOVA). A significant difference in the evolution over time of the gene expression levels between the sessile group and the planktonic group was tested with a two-way ANOVA. When the one-way ANOVA was significant, two-sided univariate tests with a correction for multiple comparisons were done (Bonferroni test) to locate the significant differences.

For the in vivo data, a one-way ANOVA was used to test if there was a significant evolution of the expression levels over time. When the one-way ANOVA test was significant, the two-sided Bonferroni multiple-comparison method was used to determine which time points differed at a=0.05, with a correction for multiple comparisons.

### Expression of the SE1501 Gene In Vitro in 0.9% NaCl.

In planktonic bacteria, expression of this gene decreased after inoculation and stayed at a low level throughout the observation period. In sessile bacteria, gene expression rapidly increased and remained at a high level.

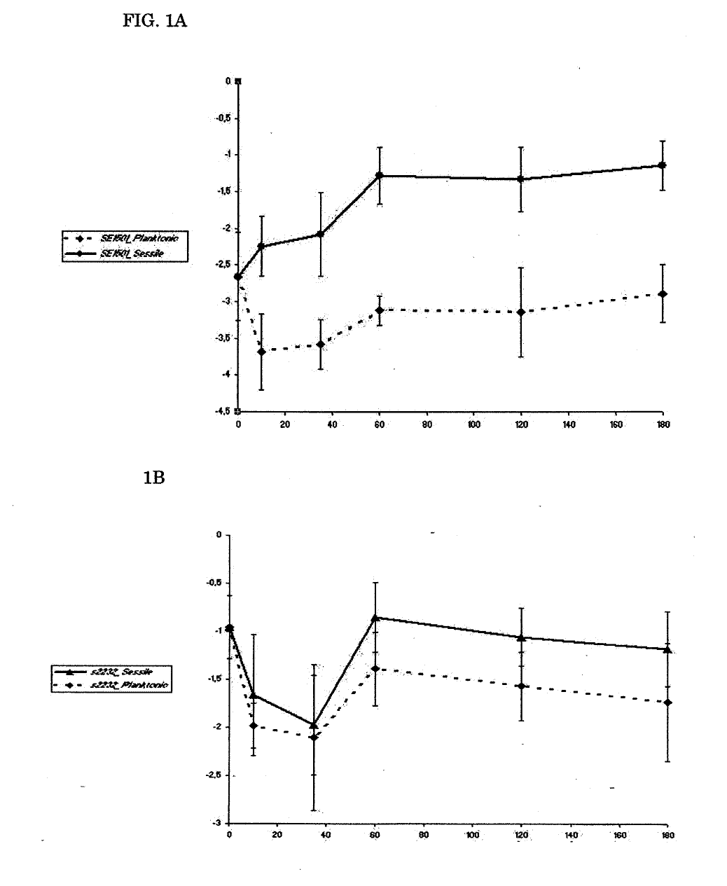

The difference between expression in sessile and planktonic bacteria was highly significant (P<0.0001, as determined by two-way ANOVA). The maximum difference in expression was reached at 60 min after inoculation (1.844 log₁₀ or 70-fold difference) and stayed almost constant till the end of the experiment (FIG. 1.A).

### Expression of the SE1501 Gene In Vivo.

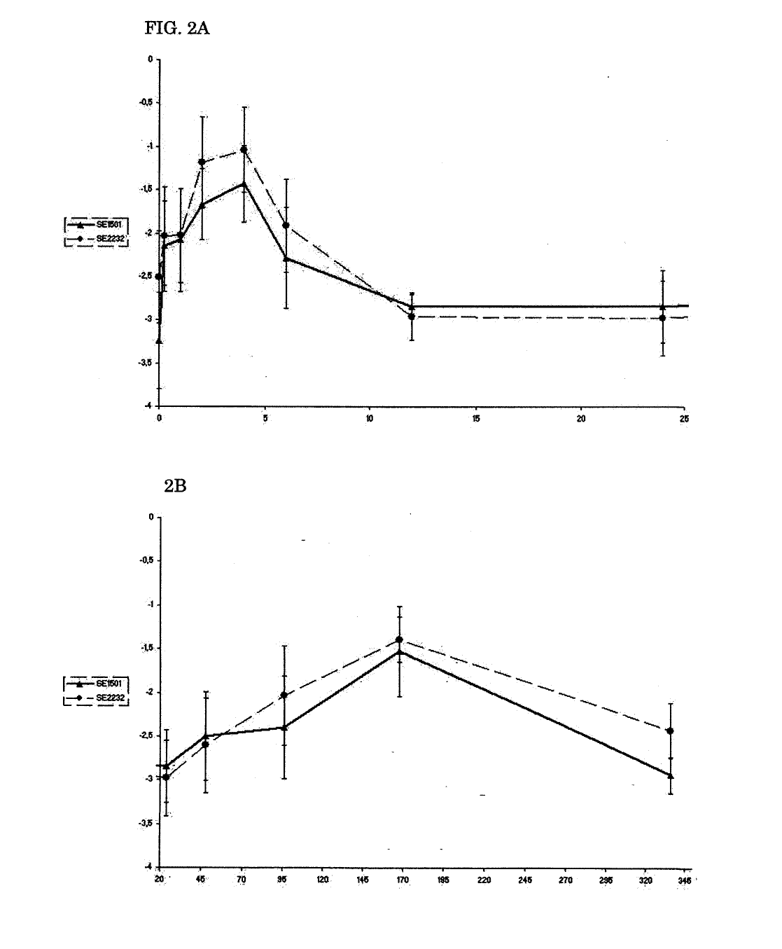

After implantation of the catheter fragments in the rats, expression in the sessile bacteria rapidly increased and peaked after 4 h (65-fold increase (95% CI of diff. −2.688 log₁₀ to −0.9352 log₁₀; P<0.001, as determined by a Bonferroni test) followed by a 26-fold decrease of gene expression in the next 20 hours. From 24 till 96 hours after implantation, a slight increase was observed which peaked at 168 h (one week) after implantation and subsequently decreased to the same level of gene expression observed at 24 h (FIG. 2).

### Expression of the SE2232 Gene In Vitro in 0.9% NaCl.

In sessile and planktonic bacteria, expression of this gene decreased 10-fold during the first 35 min after inoculation in 0.9% NaCl (P<0.01, one-way ANOVA) and then remained almost constant till the end at 180 min in planktonic bacteria. In sessile bacteria, expression of this gene increased 13-fold from 35 min to 60 min and then remained constant during the 3 h observation period of the experiment (FIG. 1.B). The gene expression difference over time between planktonic and sessile was not significant

### Expression of the SE2232 Gene In Vivo.

Implantation induced a 1.321 log₁₀ or 21-fold increase in gene expression that peaked at 4 h (1.47 log₁₀ or 30-fold increase, 95% CI of diff. −2.170 log₁₀ to −0.7704 log₁₀; P<0.001, as determined by a Bonferroni test) followed by a 1.941 log₁₀ or 87-fold decrease of gene expression in the next 20 hours. After 24 hrs, gene expression again slowly increased, peaked again at 168 hours (one week) after implantation and from then on, slowly decreased till the end at 336 h (P<0.0001, 1-way ANOVA) (FIG. 2).

Analysis of gene expression of the two genes (SE1501 and SE2232) selected for this study over time in our in vivo and in vitro models and FACS analysis and microscopy data (see Example 2) show that these two proteins are expressed in S. epidermidis 10b and are located at least partially on the surface of bacterial cells. The pattern of changes of gene expression over time in in vitro experiments is similar to the pattern of protein expression changes at relevant time points in vitro as confirmed by fluorescence microscopy data.

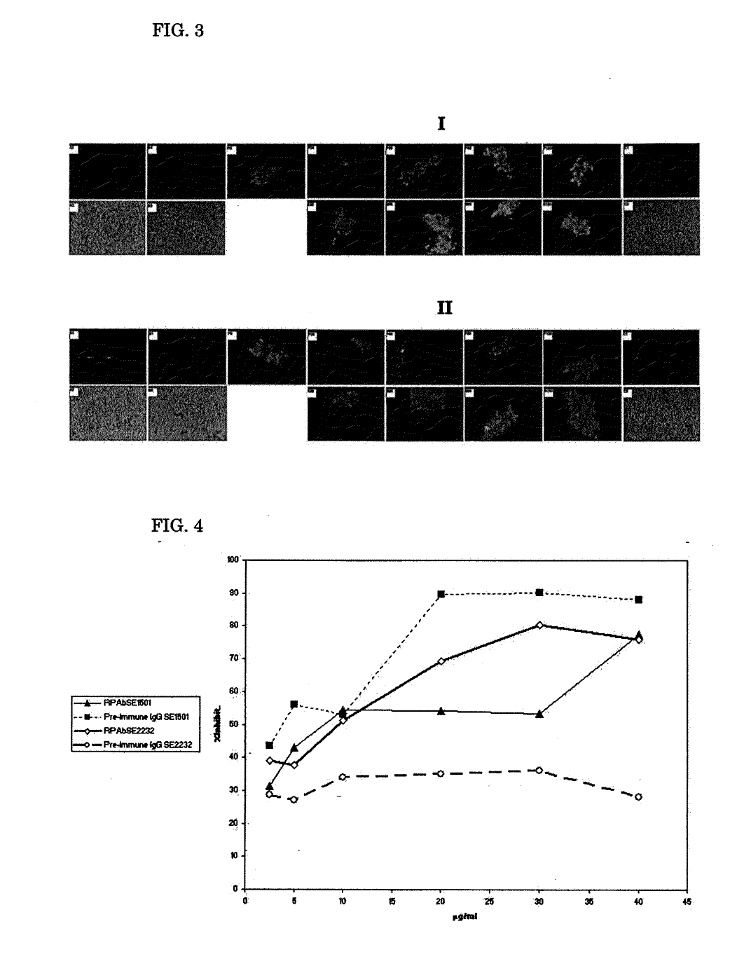

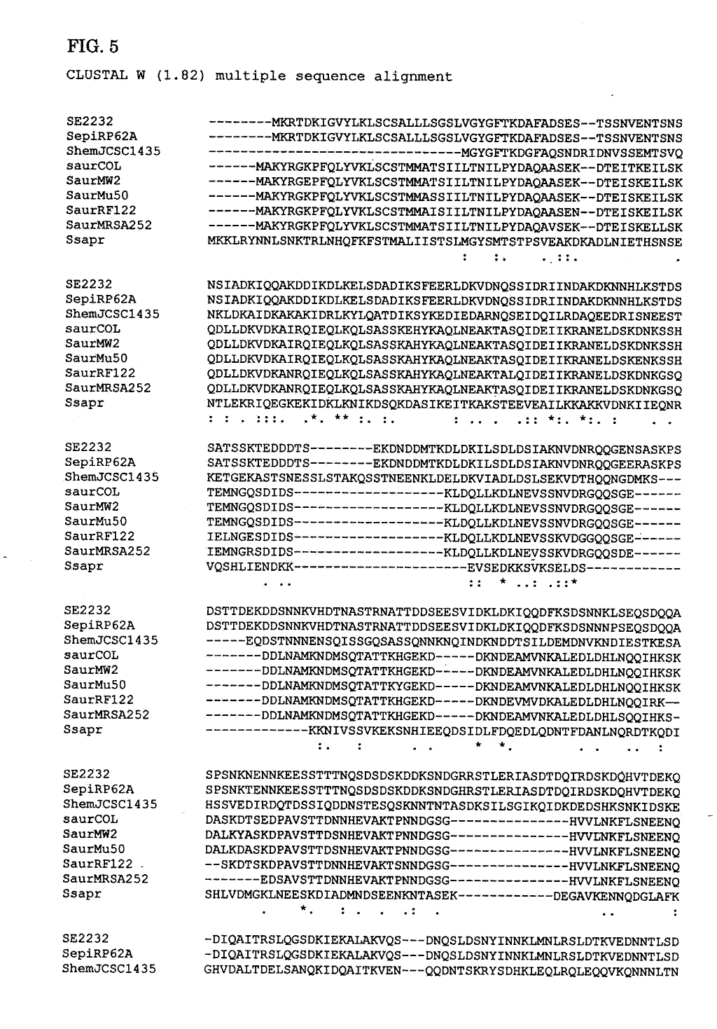

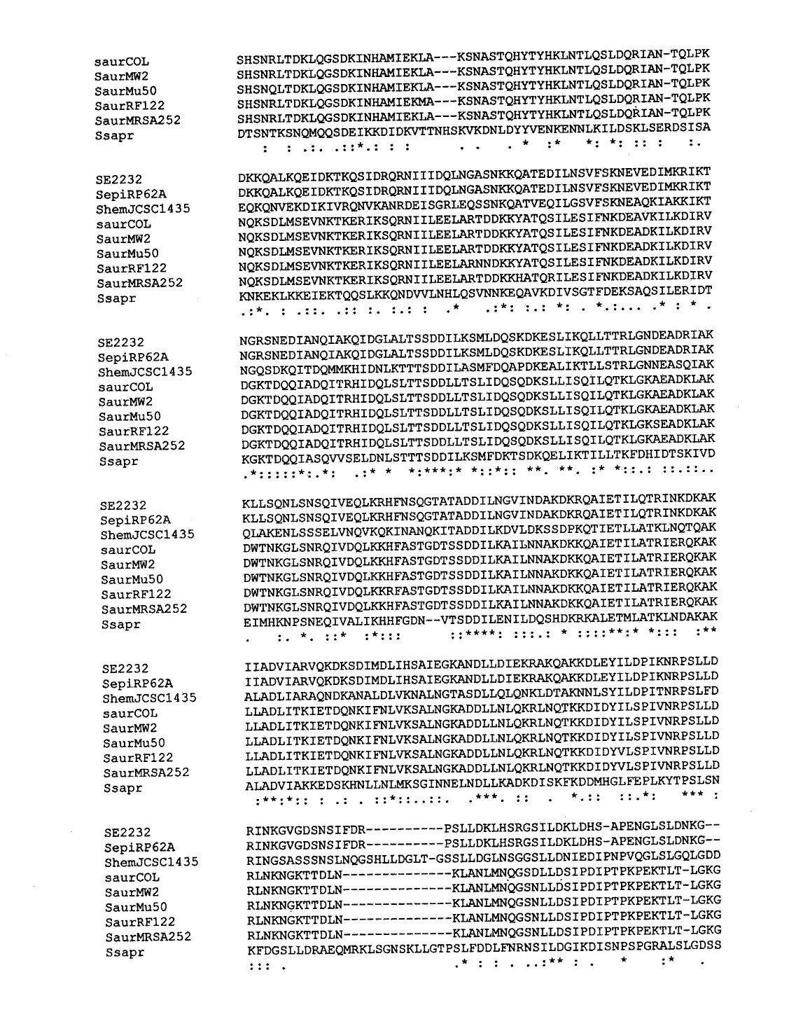

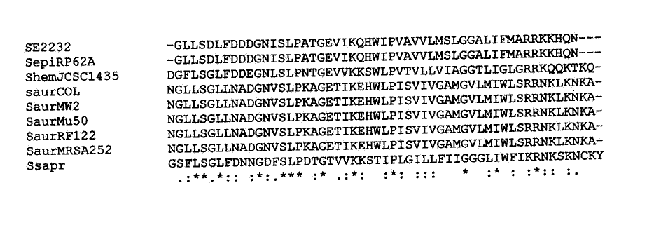

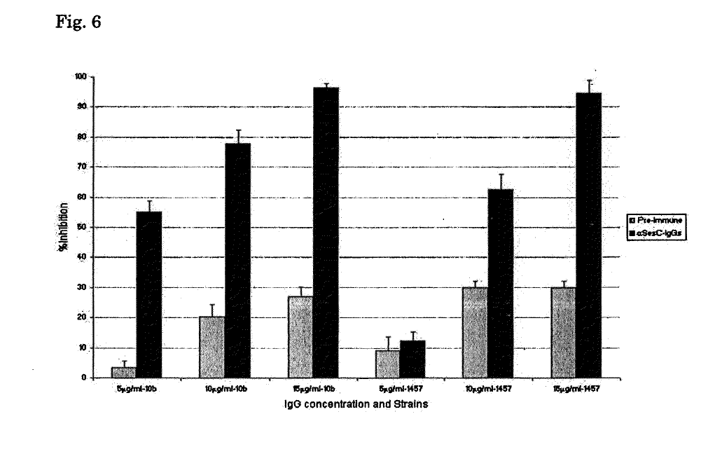

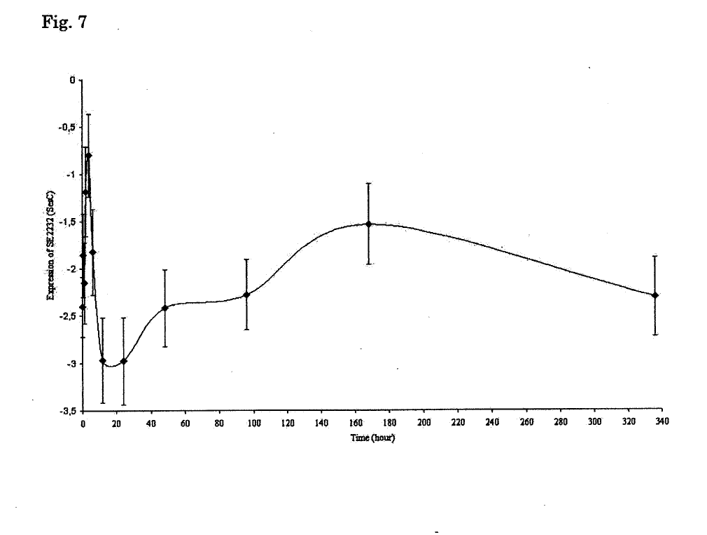

Gene expression of SE1501 slowly increased after exposure of bacteria to the foreign body and peaked 1 hour after inoculation in vitro and stayed high and constant after that. There is significant difference between the gene expression of SE1501 in sessile and planktonic bacteria. Early in vivo FBI data also showed an increase in gene expression after implantation. Microscopical images show that this protein is more expressed in sessile cells than planktonic bacteria (FIG. 3A). Since this gene is absent in the biofilm producing strain S. epidermidis RP62A (28) and since polyclonal antibodies against SE1501 didn't show any biofilm inhibition effect (our data), the role of protein might be indirect (e.g., as a signal transduction factor).

The SE2232 gene exhibits some similarity to fibronectin binding proteins such as atlC and AAS found in other Staphylococcal species which their roles in biofilm formation were proven previously. The in vivo and in vitro expression of this gene did not show any changes during the first hour. No significant difference in expression of the SE2232 gene between sessile and planktonic bacteria was observed, although from the gene expression curve and microscopy images it can be concluded that the expression of this protein in sessile bacteria is slightly higher than in planktonic bacteria (FIG. 3B). However, both SE1501 and SE2232 gene expression during late in vivo FBI were high.

### Example 2

### Production of Antibodies

### Cloning, Expression and Purification of Recombinant Proteins.

We amplified the genes for production of the recombinant proteins by PCR with primers of SE1501F (5′ CACGTGCTAGCGCTGAATCAAACACTTCAGTTTCTTCT 3′), SE1501R (5′ATGCGGATCCTAGTGATGGTGATGGTGATGCATACCTGTATTTGGTAAT AG 3′), SE2232F (5′ ACGTGCTAGCGCAGATTCAGAAAGTACATC 3′) and SE2232R (5′ATGCGGATCCTAGTGATGGTGATGGTGATGATCAGCTGTAGCTGTTCC 3′), which incorporate flanking NheI and BamHI restriction sites and sequence coding for a C-terminal His⁶ tag. The genes were cloned into a pET11c expression vector (Stratagene, LaJolla, Calif.) and we electrotransformed the recombinant plasmids into E. coli BL21 (DE3). The expression of recombinant protein was induced by the addition of IPTG, and the recombinant proteins were purified by Ni⁺ affinity chromatography with the HisTrap™ Kit (Amersham Pharmacia, Uppsala, Sweden) according to the manufactures recommendations. We determined the purity of the recombinant proteins by Coomassie Blue staining of SDS-PAGE gel electrophoresis. The sequence of the purified peptides was confirmed with MALDI-TOF mass spectrometry.

### Production of Recombinant Protein Rabbit Antiserum.

Rabbit immunization with each recombinant protein was done by Eurogentec (Seraing, Belgium), according to standard immunization protocols.

### Purification of Antibodies:

Sera from pre and post-immunization rabbits were passed onto a protein-G sepharose column (Pharmacia) equilibrated against PBS. After washing the column with PBS, bound IgG's were eluted using 0.1 M Glycin-HCl pH 2.7. The eluate was dialysed extensively against PBS and the IgG content was measured spectrophotometrically (OD₂₈₀ₙₘ, ε: 1.4).

### Flow Cytometry Analysis and Fluorescence Microscopy.

Preparation of samples for FACS analysis and fluorescence microscopy was similar to preparation of samples for gene expression analysis in vitro with the exception that here samples were taken at different time points (0, 30, 60, 90 and 120 min) of bacterial growth and planktonic cells were also sonicated using an ultrasound bath (Branson 2510), in a manner similar to that used to separate the sessile cells from the catheters. Then the bacteria were fixed using PBS containing 3% formaldehyde and 1% glutaraldehyde for 30 min to 1 hour, after which the cells were washed with PBS. The number of bacteria per ml was estimated by measuring the OD₆₀₀ₙₘ and the bacterial suspensions were subsequently diluted to an OD₆₀₀ₙₘ of 0.05 (˜5×10⁷ cells) in PBS. Purified rabbit antibodies were added at 5 μg/100 μl to the bacterial solution which was then incubated for 30 minutes on ice. Afterwards the bacteria were washed twice with PBS and FITC-labelled goat-anti-rabbit (BD-Pharmingen) was added for 20-30 min. The cells were washed twice with PBS prior to analyzing via FACS vantage (Beckton Dickinson).

The rabbit antibodies used were: RPAbSE1501 and RPAbSE2232 and a control rabbit IgG (polyclonal rabbit antibody against mouse immunoglobins). A total of 10,000 bacteria were counted and analyzed with CellQuest software (BD Biosciences).

Five μl of the solution was applied to a microscope slide and fixed with a cover glass. Cells were viewed with a fluorescence microscope (Leica, Germany) equipped with an oil-immersion plan neofluar objective (100×; numeric aperture=1.25), digital images were obtained by using corresponding filter and camera

Cells plus control rabbit IgG and FITC-labelled goat-anti-rabbit, cells plus FITC-labelled goat-anti-rabbit and cells plus respectively RPAbSE1501 or RPAbSE2232 were used as negative controls in FACS and microscopy analysis assays (FIG. 3).

### FACS Analysis.

According to data obtained by FACS analysis, the shift in fluorescence intensity was similar between sessile and planktonic cells. These results are in contradiction with the gene expression data that showed a higher level of expression of these proteins in sessile bacteria. Because this discrepancy between the FACS results and gene-expression data could be due to aggregation of sessile bacteria, we used microscope analysis to visualize the cells. We observed that sonification of sessile bacteria, even for 10 min, would not completely separate the bacteria in sessile form. Moreover, in the FACS analysis, the size effect (aggregation of cells) plays a role in the final result. In this context, it is important to mention that we observed a shift in the amount of cells from the area that cells were gated to calculate the mean of the peak toward the area of bigger particles size in sessile samples (data not shown).

### Fluorescence Microscopy and Protein Expression

Comparison of the images taken by fluorescence microscope from sessile and planktonic bacteria at different time points using RPAbSE1501 and RPAbSE2232 antibodies and FITC-labelled goat-anti-rabbit antibody confirmed the higher levels of gene expression in the sessile form (FIG. 3). To confirm that SE1501 and SE2232 proteins were localized at least partially on the surface of bacteria, bacteria from time point zero samples were incubated with primary antibodies (RPAbSE1501 or RPAbSE2232), fixed and then incubated with FITC-labelled goat-anti-rabbit antibody. The results obtained from live or fixed cells at time point zero were similar. Antibodies, being large proteins, cannot pass through intact cellular or subcellular membranes in living cells (39). Hence, it can be concluded that these two proteins are mainly located on the cell surface of bacteria.

### Example 3

### Biofilm Inhibition

### Biofilm Inhibition Assay.

The amount of biofilm formed was determined by a semiquantitive microtiter plate method as described (18, 38). A frozen culture of S. epidermidis 10b was grown to the end-exponential growth phase, pelleted, and resuspended in 0.9% NaCl, diluted to an OD₆₀₀ of 0.05 (˜5×10⁷ cells) and mixed with polyclonal antibodies. Sixplicate samples (each containing 0.1 ml) were added to individual wells of a 96-well flat-bottom microwell plate (comp). Microwell plates were incubated 2 h at 4° C. and then overnight at 37° C. After three washes with PBS, any remaining biofilm was stained with safranin O dye for 1 min and washed with PBS again. Optical density at 492 nm (OD₄₉₂) was determined with a 96-well plate spectrometer reader. Percent inhibition of biofilm formation was calculated by using the following formula: (A_{492, positive}−A_{492, antibody})/(A_{492, positive}−A_{492, negative})×100%. (18). 0.9% NaCl medium without bacteria was used as negative control.

For the biofilm inhibition data, two hypotheses were tested. A significant change in biofilm inhibition levels over different concentrations of IgG's within one group (pre or post-immunization) was tested with a one-way analysis of variance (One-way ANOVA). A significant difference in the evolution over different concentrations of IgG's of the biofilm inhibition levels between the pre-immunization group and post-immunization group was tested with a two-way ANOVA. When the one-way ANOVA was significant, two-sided univariate tests with a correction for multiple comparisons were done (Bonferroni test) to locate the significant differences.

### Biofilm Inhibition by Polyclonal Antibodies (FIG. 4).

Different concentrations of purified IgG's from pre and post-immunization sera of rabbits immunized against both SE1501 and SE2232 recombinant proteins were used to study the effect on biofilm formation.

Increasing concentrations of polyclonal RPAbSE2232 antibodies led to increasing inhibition of biofilm formation. At a concentration of 30 μg/ml, biofilm inhibition by RPAbSE2232 reached a maximum of 80%, whereas biofilm inhibition obtained by different concentrations of the IgG fraction of pre-immune rabbit sera was constant (≈30%).

Biofilm inhibition by the IgG fraction purified from pre-immune sera of rabbits subsequently immunized against SE1501 was higher than that obtained by the post-immunization IgG fraction. At a concentration of 20 μg/ml, biofilm inhibition by pre-immune IgG reached a maximum of 90%, whereas for the post-immunization IgG fraction, a maximum biofilm inhibition of 75% was obtained at a concentration of 40 μg/ml. The 75% biofilm inhibition obtained at the concentration of 40 μg/ml of post-immunization IgG is probably not a specific effect of anti-SE1501 IgG's but mainly due to antibodies against other antigens which exist in pre-immune rabbit sera.

In a second experiment a comparison of the effects of biofilm inhibition in two strong biofilm-forming Staphylococcus epidermidis strains 10b and 1457 by rabbit polyclonal anti-SesC IgGs and IgGs purified from pre-immune rabbit serum was made. The total IgG fractions from antiserum as well as pre-immune serum were purified by protein G Sepharose chromatography. The polyclonal anti-SesC IgGs were isolated by antigen specific affinity purification. The amount of biofilm formed was determined by a semi-quantitative microtiter plate method as described previously (Christensen et al., Infection and Immunity 37, 318-326). Percent inhibition of biofilm formation was calculated by using the following formula: (A_{595, positive}−A_{595, antibody})/(A_{595, positive}−A_{595, negative})×100% (Sun et al., 2005). S. epidermidis strains in brain heart infusion broth were used as the positive controls and BHI without bacteria was used as the negative control. Increasing the concentration of polyclonal anti-SesC IgGs resulted in increasing inhibition of biofilm formation up to 96% (see FIG. 6). Maximally 30% inhibition of biofilm formation by pre-immune IgGs can be considered either as background or as effect of other serum IgGs against other surface antigens of S. epidermidis.

### Example 4

### Study of the Effect of Anti-SE2232 Antibodies in the Prevention of Biofilm Formation in an In Vivo Rat Model of Foreign Body Infection

1) Construction of Isogenic S. epidermidis Strains with/without SesC

A 451 amino acid gene fragment of sesC, encoding the periplasmic part was amplified by PCR using primers incorporating flanking NheI and BamHI restriction sites and a C-terminal His⁶ tag encoding sequence. This fragment was cloned into a pET11c expression vector and electrotransformed into E. coli BL21. Expression of the recombinant protein was induced and the protein was purified by Ni⁺ affinity chromatography with the HisTrap™ Kit (Amersham Pharmacia). Purity of the recombinant protein was checked by Coomassie Blue staining of SDS-PAGE gel electrophoresis and the sequence of the purified peptide was confirmed with MALDI-TOF mass spectrometry.

2) In Vivo Rat Model of Foreign Body Infection

A rat model for foreign body infections was developed and validated in our lab (Van Wijngaerden et al., 1999, J. Antimicrob Chemother. 44:669-674; Vandecasteele et al., 2001, J. Bacteriol. 183:7094-7101). Catheter fragments, pre-incubated with bacteria are implanted subcutaneously in each rat and explanted over a period of maximum 2 weeks. Biofilm formation can be evaluated by measuring the number of copies of gmk genomic DNA recovered from each catheter segment via quantitative PCR or via determination of the number of colony-forming units by culture (see FIG. 7). As previously demonstrated the number of gmk gDNA copies per catheter correlates very well with the number of CFU per catheter. In previous experiments, we established that the efficiency of biofilm formation is strain-dependent and reaches 100% in some strains (for example S. epidermidis strain 10b—a strain isolated from a clinical case of catheter infection). We also found that antibiotic treatment can significantly affect the efficiency of biofilm formation (refs).

3) Planned Experiment and Primary Endpoints

Rat immunization with the SesC recombinant protein will be according to standard immunization protocols. Briefly, 2 groups of each 12 ex-germfree Fisher rats are used. Pre-immune serum is collected prior to the first immunization from all rats. In the test group, each rat is immunized with 100 μg SesC recombinant protein in Complete Freund's Adjuvant (CFA) intraperitoneally. Two subsequent intraperitoneal injections with 50 μg SesC recombinant protein in Incomplete Freund's Adjuvant (IFA) are given after 4 and 6 weeks. The control group receives injections with sterile saline and adjuvant.

To check the effectiveness of immunization, the pre-immune serum and post-immunization serum collected after the third injection are tested for the presence of anti-SesC antibodies using an ELISA and Western blotting.

After confirmation of the presence of anti-SesC antibodies in the test group, contaminated catheters are implanted in the rats from both groups as described. Catheters from 4 rats in each group are explanted after 2, 8 and 12 days and formation of biofilms is determined. Primary outcome measures are the number of infected catheters (>100 CFU/catheter) in each group and the number of bacteria on each catheter. The rats vaccinated with the recombinant protein show a decrease in both these measures as compared to control.

1. A method for preventing biofilm formation by Staphylococcus on a medical device comprising coating said medical device with an antibody against protein SE2232 (sesC) of Staphylococcus epidermis or a protein that has more than 70% identity therewith.

2. A method according to claim 1, wherein said medical device is an intravascular device such as a stent or a cannula, a prosthesis, such as an orthopaedic endoprothesis or a prosthetic valve, a pacemaker, a catheter, a drain, an endovascular graft or a cerebrovascular shunt.

3. A method according to claim 1 or 2, wherein said coated medical device is implanted or inserted into a mammalian body, preferably a human body.

4. Antibody against protein SE2232 of Staphylococcus epidermis or a protein that has more than 70% identity therewith.

5. Antibody according to claim 2, selected from the group consisting of polyclonal antibodies, monoclonal antibodies, single chain antibodies, humanised antibodies and SE2232 binding antibody fragments such as F(ab), F(ab)2 and scFV.

6. Antibody according to claim 2, where the protein is chosen from the group of proteins listed in SEQ ID NO 24, 25, 26, 27, 28, 29, 30, 31 and 32.

7. Immunogenic composition comprising protein SE2232 of Staphylococcus epidermis, a protein that has more than 70% identity therewith, or an immunologically active fragment of any of those.

8. Immunogenic composition according to claim 7, wherein said immunologically active fragment is the 451 amino acids fragment flanking the NheI and BamHI restriction sites.

9. Vaccine composition comprising the immunogenic composition of claim 7 or 8 and a pharmaceutically acceptable carrier.

10. A method for preventing biofilm formation in a mammal by administering an antibody according to any of the claims 4-6 or a vaccine composition according to claim 9 to a subject in need thereof.

11. A method according to claim 10, wherein said method comprises a prophylactic or a therapeutical treatment.

12. Use of an antibody according to any of claims 4-6 or a vaccine composition according to claim 9 for preventing biofilm formation.

13. Use according to claim 12, wherein the antibody is coated onto a medical device.

14. A medical device coated with an antibody according to any of claims 4-6.


SEQUENCE LISTING (32 sequences)

SEQ ID NO: 1 - protein, 5 aa, Artificial
  LPxTG motif
LPXTG

SEQ ID NO: 2 - protein, 15 aa, Artificial
  linker 1
GGGGSGGGGSGGGGS

SEQ ID NO: 3 - protein, 15 aa, Artificial
  linker 2
ESGRSGGGGSGGGGS

SEQ ID NO: 4 - protein, 14 aa, Artificial
  linker 3
EGKSSGSGSESKST

SEQ ID NO: 5 - protein, 15 aa, Artificial
  linker 4
EGKSSGSGSESKSTQ

SEQ ID NO: 6 - protein, 14 aa, Artificial
  linker 5
EGKSSGSGSESKVD

SEQ ID NO: 7 - protein, 14 aa, Artificial
  linker 6
GSTSGSGKSSEGKG

SEQ ID NO: 8 - protein, 18 aa, Artificial
  linker 7
KESGSVSSEQLAQFRSLD

SEQ ID NO: 9 - protein, 16 aa, Artificial
  linker 8
ESGSVSSEELAFRSLD

SEQ ID NO: 10 - DNA, 23 bp, Artificial
  Fd primer 1501
ccaattactagtattaaattcag

SEQ ID NO: 11 - DNA, 19 bp, Artificial
  Rv primer 1501
ctacactgttagacgtgag

SEQ ID NO: 12 - DNA, 20 bp, Artificial
  Fd primer 2232
gttgataaccgtcaacaagg

SEQ ID NO: 13 - DNA, 21 bp, Artificial
  Rv primer 2232
catgttgatcttttgaatccc

SEQ ID NO: 14 - DNA, 28 bp, Artificial
  TFd primer 1501
ccctaaagatgaaactactgttcatgat

SEQ ID NO: 15 - DNA, 36 bp, Artificial
  1501 probe
acaatgtgacaaaacagggtgtactaaaagtaaacg

SEQ ID NO: 16 - DNA, 26 bp, Artificial
  TRv primer 1501
tgctatccattcgaaactttcattat

SEQ ID NO: 17 - DNA, 26 bp, Artificial
  TFd primer 2232
agcatcaccatctaataaaaacgaaa

SEQ ID NO: 18 - DNA, 39 bp, Artificial
  2232 probe
taacaaagaagaatctagtacgacaacaaatcaatccga

SEQ ID NO: 19 - DNA, 31 bp, Artificial
  TRv primer 2232
ccatcattacttttatcgtctttactatcac

SEQ ID NO: 20 - DNA, 38 bp, Artificial
  SE1501F primer
cacgtgctagcgctgaatcaaacacttcagtttcttct

SEQ ID NO: 21 - DNA, 51 bp, Artificial
  SE1501R primer
atgcggatcctagtgatggtgatggtgatgcatacctgtatttggtaatag

SEQ ID NO: 22 - DNA, 30 bp, Artificial
  SE2232F primer
acgtgctagcgcagattcagaaagtacatc

SEQ ID NO: 23 - DNA, 48 bp, Artificial
  SE2232R primer
atgcggatcctagtgatggtgatggtgatgatcagctgtagctgttcc

SEQ ID NO: 24 - protein, 676 aa, Staphylococcus epidermidis
MKRTDKIGVYLKLSCSALLLSGSLVGYGFTKDAFADSESTSSNVENTSNSNSIADKIQQAKDDIKDLKELSDADIKSFEERLDKVDNQSSIDRIINDAKDKNNHLKSTDSSATSSKTEDDDTSEKDNDDMTKDLDKILSDLDSIAKNVDNRQQGENSASKPSDSTTDEKDDSNNKVHDTNASTRNATTDDSEESVIDKLDKIQQDFKSDSNNKLSEQSDQQASPSNKNENNKEESSTTTNQSDSDSKDDKSNDGRRSTLERIASDTDQIRDSKDQHVTDEKQDIQAITRSLQGSDKIEKALAKVQSDNQSLDSNYINNKLMNLRSLDTKVEDNNTLSDDKKQALKQEIDKTKQSIDRQRNIIIDQLNGASNKKQATEDILNSVFSKNEVEDIMKRIKTNGRSNEDIANQIAKQIDGLALTSSDDILKSMLDQSKDKESLIKQLLTTRLGNDEADRIAKKLLSQNLSNSQIVEQLKRHFNSQGTATADDILNGVINDAKDKRQAIETILQTRINKDKAKIIADVIARVQKDKSDIMDLIHSAIEGKANDLLDIEKRAKQAKKDLEYILDPIKNRPSLLDRINKGVGDSNSIFDRPSLLDKLHSRGSILDKLDHSAPENGLSLDNKGGLLSDLFDDDGNISLPATGEVIKQHWIPVAVVLMSLGGALIFMARRKKHQN

SEQ ID NO: 25 - protein, 676 aa, Staphylococcus epidermidis
MKRTDKIGVYLKLSCSALLLSGSLVGYGFTKDAFADSESTSSNVENTSNSNSIADKIQQAKDDIKDLKELSDADIKSFEERLDKVDNQSSIDRIINDAKDKNNHLKSTDSSATSSKTEDDDTSEKDNDDMTKDLDKILSDLDSIAKNVDNRQQGEERASKPSDSTTDEKDDSNNKVHDTNASTRNATTDDSEESVIDKLDKIQQDFKSDSNNNPSEQSDQQASPSNKTENNKEESSTTTNQSDSDSKDDKSNDGHRSTLERIASDTDQIRDSKDQHVTDEKQDIQAITRSLQGSDKIEKALAKVQSDNQSLDSNYINNKLMNLRSLDTKVEDNNTLSDDKKQALKQEIDKTKQSIDRQRNIIIDQLNGASNKKQATEDILNSVFSKNEVEDIMKRIKTNGRSNEDIANQIAKQIDGLALTSSDDILKSMLDQSKDKESLIKQLLTTRLGNDEADRIAKKLLSQNLSNSQIVEQLKRHFNSQGTATADDILNGVINDAKDKRQAIETILQTRINKDKAKIIADVIARVQKDKSDIMDLIHSAIEGKANDLLDIEKRAKQAKKDLEYILDPIKNRPSLLDRINKGVGDSNSIFDRPSLLDKLHSRGSILDKLDHSAPENGLSLDNKGGLLSDLFDDDGNISLPATGEVIKQHWIPVAVVLMSLGGALIFMARRKKHQN

SEQ ID NO: 26 - protein, 670 aa, Staphylococcus haemolyticus
MGYGFTKDGFAQSNDRIDNVSSEMTSVQNKLDKAIDKAKAKIDRLKYLQATDIKSYKEDIEDARNQSEIDQILRDAQEEDRISNEESTKETGEKASTSNESSLSTAKQSSTNEENKLDELDKVIADLDSLSEKVDTHQQNGDMKSEQDSTNNNENSQISSGQSASSQNNKNQINDKNDDTSILDEMDNVKNDIESTKESAHSSVEDIRDQTDSSIQDDNSTESQSKNNTNTASDKSILSGIKQIDKDEDSHKSNKIDSKEGHVDALTDELSANQKIDQAITKVENQQDNTSKRYSDHKLEQLRQLEQQVKQNNNLTNEQKQNVEKDIKIVRQNVKANRDEISGRLEQSSNKQATVEQILGSVFSKNEAQKIAKKIKTNGQSDKQITDQMMKHIDNLKTTTSDDILASMFDQAPDKEALIKTLLSTRLGNNEASQIAKQLAKENLSSSELVNQVKQKINANQKITADDILKDVLDKSSDPKQTIETLLATKLNQTQAKALADLIARAQNDKANALDLVKNALNGTASDLLQLQNKLDTAKNNLSYILDPITNRPSLFDRINGSASSSNSLNQGSHLLDGLTGSSLLDGLNSGGSLLDNIEDIPNPVQGLSLGQLGDDDGFLSGLFDDEGNLSLPNTGEVVKKSWLPVTVLLVIAGGTLIGLGRRKQQKTKQ

SEQ ID NO: 27 - protein, 635 aa, Staphylococcus aureus
MAKYRGKPFQLYVKLSCSTMMATSIILTNILPYDAQAASEKDTEITKEILSKQDLLDKVDKAIRQIEQLKQLSASSKEHYKAQLNEAKTASQIDEIIKRANELDSKDNKSSHTEMNGQSDIDSKLDQLLKDLNEVSSNVDRGQQSGEDDLNAMKNDMSQTATTKHGEKDDKNDEAMVNKALEDLDHLNQQIHKSKDASKDTSEDPAVSTTDNNHEVAKTPNNDGSGHVVLNKFLSNEENQSHSNRLTDKLQGSDKINHAMIEKLAKSNASTQHYTYHKLNTLQSLDQRIANTQLPKNQKSDLMSEVNKTKERIKSQRNIILEELARTDDKKYATQSILESIFNKDEAVKILKDIRVDGKTDQQIADQITRHIDQLSLTTSDDLLTSLIDQSQDKSLLISQILQTKLGKAEADKLAKDWTNKGLSNRQIVDQLKKHFASTGDTSSDDILKAILNNAKDKKQAIETILATRIERQKAKLLADLITKIETDQNKIFNLVKSALNGKADDLLNLQKRLNQTKKDIDYILSPIVNRPSLLDRLNKNGKTTDLNKLANLMNQGSDLLDSIPDIPTPKPEKTLTLGKGNGLLSGLLNADGNVSLPKAGETIKEHWLPISVIVGAMGVLMIWLSRRNKLKNKA

SEQ ID NO: 28 - protein, 635 aa, Staphylococcus aureus
MAKYRGEPFQLYVKLSCSTMMATSIILTNILPYDAQAASEKDTEISKEILSKQDLLDKVDKAIRQIEQLKQLSASSKAHYKAQLNEAKTASQIDEIIKRANELDSKDNKSSHTEMNGQSDIDSKLDQLLKDLNEVSSNVDRGQQSGEDDLNAMKNDMSQTATTKHGEKDDKNDEAMVNKALEDLDHLNQQIHKSKDALKYASKDPAVSTTDSNHEVAKTPNNDGSGHVVLNKFLSNEENQSHSNRLTDKLQGSDKINHAMIEKLAKSNASTQHYTYHKLNTLQSLDQRIANTQLPKNQKSDLMSEVNKTKERIKSQRNIILEELARTDDKKYATQSILESIFNKDEADKILKDIRVDGKTDQQIADQITRHIDQLSLTTSDDLLTSLIDQSQDKSLLISQILQTKLGKAEADKLAKDWTNKGLSNRQIVDQLKKHFASTGDTSSDDILKAILNNAKDKKQAIETILATRIERQKAKLLADLITKIETDQNKIFNLVKSALNGKADDLLNLQKRLNQTKKDIDYILSPIVNRPSLLDRLNKNGKTTDLNKLANLMNQGSNLLDSIPDIPTPKPEKTLTLGKGNGLLSGLLNADGNVSLPKAGETIKEHWLPISVIVGAMGVLMIWLSRRNKLKNKA

SEQ ID NO: 29 - protein, 635 aa, Staphylococcus aureus
MAKYRGKPFQLYVKLSCSTMMASSIILTNILPYDAQAASEKDTEISKEILSKQDLLDKVDKAIRQIEQLKQLSASSKAHYKAQLNEAKTASQIDEIIKRANELDSKENKSSHTEMNGQSDIDSKLDQLLKDLNEVSSNVDRGQQSGEDDLNAMKNDMSQTATTKYGEKDDKNDEAMVNKALEDLDHLNQQIHKSKDALKDASKDPAVSTTDSNHEVAKTPNNDGSGHVVLNKFLSNEENQSHSNQLTDKLQGSDKINHAMIEKLAKSNASTQHYTYHKLNTLQSLDQRIANTQLPKNQKSDLMSEVNKTKERIKSQRNIILEELARTDDKKYATQSILESIFNKDEADKILKDIRVDGKTDQQIADQITRHIDQLSLTTSDDLLTSLIDQSQDKSLLISQILQTKLGKAEADKLAKDWTNKGLSNRQIVDQLKKHFASTGDTSSDDILKAILNNAKDKKQAIETILATRIERQKAKLLADLITKIETDQNKIFNLVKSALNGKADDLLNLQKRLNQTKKDIDYILSPIVNRPSLLDRLNKNGKTTDLNKLANLMNQGSNLLDSIPDIPTPKPEKTLTLGKGNGLLSGLLNADGNVSLPKAGETIKEHWLPISVIVGAMGVLMIWLSRRNKLKNKA

SEQ ID NO: 30 - protein, 631 aa, Staphylococcus aureus
MAKYRGKPFQLYVKLSCSTMMAISIILTNILPYDAQAASENDTEISKEILSKQDLLDKVDKANRQIEQLKQLSASSKAHYKAQLNEAKTALQIDEIIKRANELDSKDNKGSQIELNGESDIDSKLDQLLKDLNEVSSKVDGGQQSGEDDLNAMKNDMSQTATTKHGEKDDKNDEVMVDKALEDLDHLNQQIRKSKDTSKDPAVSTTDNNHEVAKTSNNDGSGHVVLNKFLSNEENQSHSNRLTDKLQGSDKINHAMIEKMAKSNASTQHYTYHKLNTLQSLDQRIANTQLPKNQKSDLMSEVNKTKERIKSQRNIILEELARNNDKKYATQSILESIFNKDEADKILKDIRVDGKTDQQIADQITRHIDQLSLTTSDDLLTSLIDQSQDKSLLISQILQTKLGKSEADKLAKDWTNKGLSNRQIVDQLKKRFASTGDTSSDDILKAILNNAKDKKQAIETILATRIERQKAKLLADLITKIETDQNKIFNLVKSALNGKADDLLNLQKRLNQTKKDIDYVLSPIVNRPSLLDRLNKNGKTTDLNKLANLMNQGSNLLDSIPDIPTPKPEKTLTLGKGNGLLSGLLNADGNVSLPKAGETIKEHWLPISVIVGAMGVLMIWLSRRNKLKNKA

SEQ ID NO: 31 - protein, 627 aa, Staphylococcus aureus
MAKYRGKPFQLYVKLSCSTMMATSIILTNILPYDAQAVSEKDTEISKELLSKQDLLDKVDKANRQIEQLKQLSASSKAHYKAQLNEAKTASQIDEIIKRANELDSKDNKGSQIEMNGRSDIDSKLDQLLKDLNEVSSKVDRGQQSDEDDLNAMKNDMSQTATTKHGEKDDKNDEAMVNKALEDLDHLSQQIHKSEDSAVSTTDNNHEVAKTPNNDGSGHVVLNKFLSNEENQSHSNRLTDKLQGSDKINHAMIEKLAKSNASTQHYTYHKLNTLQSLDQRIANTQLPKNQKSDLMSEVNKTKERIKSQRNIILEELARTDDKKHATQRILESIFNKDEADKILKDIRVDGKTDQQIADQITRHIDQLSLTTSDDLLTSLIDQSQDKSLLISQILQTKLGKAEADKLAKDWTNKGLSNRQIVDQLKKHFASTGDTSSDDILKAILNNAKDKKQAIETILATRIERQKAKLLADLITKIETDQNKIFNLVKSALNGKADDLLNLQKRLNQTKKDIDYVLSPIVNRPSLLDRLNKNGKTTDLNKLANLMNQGSNLLDSIPDIPTPKPEKTLTLGKGNGLLSGLLNADGNVSLPKAGETIKEHWLPISVIVGAMGVLMIWLSRRNKLKNKA

SEQ ID NO: 32 - protein, 654 aa, Staphylococcus saprophyticus
MKKLRYNNLSNKTRLNHQFKFSTMALIISTSLMGYSMTSTPSVEAKDKADLNIETHSNSENTLEKRIQEGKEKIDKLKNIKDSQKDASIKEITKAKSTEEVEAILKKAKKVDNKIIEQNRVQSHLIENDKKEVSEDKKSVKSELDSKKNIVSSVKEKSNHIEEQDSIDLFDQEDLQDNTFDANLNQRDTKQDISHLVDMGKLNEESKDIADMNDSEENKNTASEKDEGAVKENNQDGLAFKDTSNTKSNQMQQSDEIKKDIDKVTTNHSKVKDNLDYYVENKENNLKILDSKLSERDSISAKNKEKLKKEIEKTQQSLKKQNDVVLNHLQSVNNKEQAVKDIVSGTFDEKSAQSILERIDTKGKTDQQIASQVVSELDNLSTTTSDDILKSMFDKTSDKQELIKTILLTKFDHIDTSKIVDEIMHKNPSNEQIVALIKHHFGDNVTSDDILENILDQSHDKRKALETMLATKLNDAKAKALADVIAKKEDSKHNLLNLMKSGINNELNDLLKADKDISKFKDDMHGLFEPLKYTPSLSNKFDGSLLDRAEQMRKLSGNSKLLGTPSLFDDLFNRNSILDGIKDISNPSPGRALSLGDSSGSFLSGLFDNNGDFSLPDTGTVVKKSTIPLGILLFIIGGGLIWFIKRNKSKNCKY